(19) United States
(12) Patent Application Publication
(10) Pub. No.: US 2012/0189595 A1
(43) Pub. Date: Jul. 26, 2012

(54) INDUCED PLURIPOTENT STEM CELLS AND METHODS OF USE

(75) Inventors: Angel Raya, Barcelona (ES); Trond Aasen, Barcelona (ES); Juan Carlos Izpisua Belmonte, Barcelona (ES)

(73) Assignee: SALK INSTITUTE FOR BIOLOGICAL STUDIES, La Jolla, CA (US)

(21) Appl. No.: 13/120,727

(22) PCT Filed: Sep. 25, 2009
(86) PCT No.: PCT/US2009/058424

§ 371 Date: Mar. 19, 2012

Related U.S. Application Data
(60) Provisional application No. 61/100,110, filed on Sep. 25, 2008.

(51) Int. Cl. A61K 35/12; C12N 15/867; A61P 17/00; C12N 5/10; C12N 15/85

(52) U.S. Cl. 424/93.21; 435/325; 435/371; 435/455; 435/456

(57) ABSTRACT

The invention relates to the field of stem cells and, specially, to the reprogramming of adult somatic cells; to obtain pluripotent cells by the transfection of specific genes. Thus, the invention provides induced pluripotent stem cells (iPS) and methods of obtaining and using them.

### RELATED APPLICATION

This application claims priority to U.S. provisional application Ser. No. 61/100,110, filed Sep. 25, 2008, the entire disclosure of which is incorporated herein by this reference.

### FIELD OF THE INVENTION

The present invention relates to the field of stem cells and, in particular, to the reprogramming of adult somatic cells to obtain pluripotent cells by transfection with specific genes. Thus, the present invention provides induced pluripotent stem cells (iPS) and methods for obtaining them.

### BACKGROUND OF THE INVENTION

Takahashi et al. (Cell 131, 861-872 (2007)) have disclosed methods for reprogramming differentiated cells, without the use of any embryo or ES (embryonic stem) cell, and establishing an inducible pluripotent stem cell having similar pluripotency and growing abilities to those of an ES cell. Takahashi et al. describe various different nuclear reprogramming factors for differentiated fibroblasts, which include products of the following four genes: an Oct family gene; a Sox family gene; a Klf family gene; and a Myc family gene.

The technology developed by Takahashi et al. also known as iPS cell technology, not only creates unprecedented opportunities for the generation of patient-specific pluripotent cells, but also allows the investigation of the molecular logic that underlies cellular pluripotency and reprogramming. However, both prospects are hampered by the low efficiency of the reprogramming process, which most likely depends on many factors, including age, type, and origin of the cells used. Further, the iPS technology, when applied to fibroblasts, seems to be very slow since the reprogramming time needed to obtain iPS cells usually takes more than twenty days.

### SUMMARY OF THE INVENTION

The invention relates to an isolated keratinocyte or keratinocyte population transfected with the genes selected from the group which comprises: an Oct4 family gene; Sox2 family gene and a Klf family gene.

In one embodiment, the keratinocyte or keratinocyte population is further transfected with a c-Myc family gene.

In another embodiment, the transfected genes also comprise a promoter to modulate its expression.

In another embodiment, the keratinocyte or keratinocyte population is of human origin.

In another embodiment, at least one of the genes is cloned into a murine stem cell virus (MSCV) derived retroviral vector.

The invention also relates to an isolated keratinocyte induced stem (KiPS) cell or KiPS cell population derived from an isolated keratinocyte or keratinocyte population transfected with the genes selected from the group which comprises: an Oct4 family gene; Sox2 family gene and a Klf family gene.

In one embodiment, the keratinocyte or keratinocyte population is further transfected with a c-Myc family gene.

In another embodiment, the transfected genes also comprise a promoter to modulate its expression.

In another embodiment, the keratinocyte or keratinocyte population is of human origin.

In another embodiment, at least one of the genes is cloned into a murine stem cell virus (MSCV) derived retroviral vector.

In another embodiment, the KiPS cell or KiPS cell population is characterized by the expression of one or more of the following markers Nanog, Oct 4, Sox2, Rex1, Cripto, Connexin43, IGF-1 receptor, SSEA4, SSEA3, Tra-1-61 and Tra-1-81.

In another embodiment, the KiPS cell or KiPS cell population is further characterized by the expression of one or more of the following markers: AP marker, CD24, CD90, CD29, CD9 and CD49f.

The invention also relates to a method for obtaining a KiPS cell or a KiPS cell population according to the invention, which comprises the transfection of a polynucleotide or polynucleotides into an isolated keratinocyte or isolated keratinocyte population, wherein the polynucleotide or polynucleotides encodes the following group of genes: an Oct4 family gene; a Sox2 family gene, and a Klf family gene.

In one embodiment, the polynucleotide or polynucleotides further encodes a c-Myc family gene.

In another embodiment, the polynucleotide or polynucleotides are cloned into a vector.

In one embodiment, the vector is the murine stem cell virus (MSCV) derived retroviral vector.

In one embodiment the isolated keratinocyte or isolated keratinocyte population are obtained by plating a hair or a root hair in a culture media which promotes keratinocyte proliferation.

The invention also relates to a method for obtaining iPS cells which comprises the steps of
a. Positively selecting an isolated somatic cell or an isolated somatic cell population by the comparison of the expression level of any of the markers c-Myc or Klf4 with an isolated fibroblast cell or an isolated fibroblast population, wherein said positive selection is made, where the expression level of either of said markers is at least 10 fold higher in comparison with the expression level in the isolated fibroblast cell or the isolated fibroblast population,
b. Transfecting the selected isolated somatic cell or the selected isolated somatic population selected in step a) with a polynucleotide or polynucleotides encoding the following group of genes: an Oct4 family gene; Sox2 family gene, a Klf family gene, and
c. Placing the transfected isolated somatic cell or isolated cell population in an appropriate dedifferentiating medium.

In one embodiment, the group of step b) also comprises a c-Myc family gene.

In another embodiment, the isolated somatic cell or the isolated somatic cell population is human.

The invention also relates to a composition comprising the keratinocyte population of the invention for use as a medicament.

In one embodiment, the composition is used for the regeneration of a tissue with mesenchymal origin, ectodermal origin or endodermal origin.

The invention also relates to a composition comprising the KiPS cell population of the invention for use as a medicament.

In one embodiment, the composition is used as a medicament for the treatment or regeneration of a tissue with mesenchymal origin, ectodermal origin or endodermal origin.

The invention also relates to the use of the keratinocyte or keratinocyte population of the invention for the elaboration or manufacture of a medicament.

In one embodiment, the composition of the invention is used for the elaboration or manufacture of a medicament for the treatment or regeneration of a tissue with mesenchymal origin, ectodermal origin or endodermal origin.

In another embodiment, the composition of the invention is used for the elaboration of a medicament for the treatment or regeneration of a tissue with mesenchymal origin, ectodermal origin or endodermal origin.

The invention also relates to a method of treating a patient with a defect in a tissue with mesenchymal origin, ectodermal origin or endodermal origin comprising the administration of the keratinocyte or keratinocyte population of the invention.

The invention also relates to a method of treating a patient with a defect in a tissue with mesenchymal origin, ectodermal origin or endodermal origin comprising the administration of KiPS cells or the KiPS cells population of the invention.

The invention also relates to a method of treating a patient with a defect in a tissue with mesenchymal origin, ectodermal origin or endodermal origin comprising the administration of the composition of the invention.

An aspect of the present invention relates to a new type of iPS cells, KiPS cells, which are derived from keratinocytes, preferably human keratinocytes. These KiPS cells, which are obtained by the transfection of keratinocytes, preferably human and more preferably primary human keratinocytes (transfected keratinocytes), to express the following genes or markers: an Oct4 family gene; Sox2 family gene, a Klf family gene; and, optionally, a Myc family gene. KiPS cells display typical ES cell like morphology and have a remarkable pluripotent, tripotent or totipotent capacity.

As described below, KiPS cells are capable of differentiating into the cell types derived from the three embryonic cell layers: mesoderm, endoderm or ectoderm. However, another characteristic feature of the KiPS cells of the invention is the ability to be cultured in basic medium for a prolonged period of time without differentiation occurring. Thus, the KiPS cells of the invention are able to remain in culture as undifferentiated cells through a number of passages, and do not differentiate until they are provided with appropriate differentiating media. Upon exposure to appropriate differentiating media, the KiPS cells are capable of differentiating into any cell with mesodermal, ectodermal or endodermal origin. The KiPS cells of the invention appear indistinguishable from human embryonic stem (hES) cells in colony morphology, growth properties, expression of pluripotency-associated transcription factors and surface markers, and in vitro and in vivo differentiation potential. Co-transduction with c-Myc, while not necessary for KiPS cell generation, results in a faster and more efficient process. Overall, the keratinocyte reprogramming process, when using the four factors, is, at least, 100-fold more efficient and 2-fold faster than that of fibroblasts.

A further aspect of the present invention relates to a method for obtaining KiPS cells which comprises the introduction of a polynucleotide or polynucleotides into an isolated keratinocyte or isolated keratinocyte population, said polynucleotide or polynucleotides encoding the following group of genes or markers: an Oct4 family gene; Sox2 family gene, a Klf family gene. In one embodiment of the invention the polynucleotide or polynucleotides further encodes a c-Myc family gene. With this method, the generation of iPS cells-KiPS cells-appears to be much more efficient than with other methods previously described.

Furthermore, the uses of the transfected keratinocytes, the KiPS cells and the progeny thereof also form part of the invention.

### BRIEF DESCRIPTION OF THE DRAWINGS

FIG. 1 shows: a) Morphology of keratinocytes grown in serum free medium after infection with GFP retrovirus showing expression in most cells (b) apart from terminally differentiating keratinocytes, c) small ES-like colony 10 days post-infection, d) example of a completely differentiated ball of keratinocytes 10 days post-infection, e) ES-like iPS colony 13 days post infection. f) iPS colonies positive for AP 7 days after mechanical picking (at day 21), g) representative immunofluorescence analysis of KiPS cells growing on matrigel. Quantitative FACS analysis of typical ES cell surface antigens in comparison with primary human keratinocytes (h) or with KiPS cells (i).

FIG. 2 shows: a) PCR analysis specific for retroviral DNA of Oct4, Sox2, Klf and, Myc factors in KiPS4F and KiPS3F1 cells, b) Western blot of protein levels of the specified factors in keratinocytes, KiPS cell lines, human embryonic stem cells and 293T cells, c) expression levels of transgene and endogenous factors quantified by qPCR and plotted relative to GAPDH expression correlates to Western blotting data.

FIG. 3: After generation of embryoid bodies (a), KiPS cells were specifically directed to differentiate into (b) endoderm, (c) mesoderm and (d) ectoderm. e) Specific differentiation of KiPS to dopaminergic neurons. f) Enlarged view of panel e. Spontaneous differentiation into all three germ layers was also evident in teratomas (g) including TuJ1 positive ectoderm (h), α-fetoprotein positive endoderm (i) and α-actinin positive mesoderm (j).

FIG. 4 shows: a) overview of multiple colonies 10 days post infection displaying several partial alkaline positive colonies, negative colonies and several differentiated balls of keratinocytes. Detailed examples are shown in b (10 days) and c (14 days). d-e) Completely AP positive after 17 days and 21 days respectfully; f, g, h) shows positive colonies for SSEA4 and Tra1.81 after 14 days; i) whole 10 cm dishes with keratinocytes colonies stained for AP after 14 and 21 days post infection and whole 10 cm dishes with fibroblasts colonies stained for AP after 31 days.

FIG. 5 shows Q-PCR analysis of 98 stem cell genes comparing fibroblasts, keratinocytes, ES4 cells and KiPS cells.

FIG. 6 shows: a) the internal part of a single scalp hair placed in matrigel coated 35 mm dish containing irMEF conditioned ES media; b) Outgrowth of keratinocytes from the outer root sheet (but not the hair bulb area) after 5 days. After 8 days cells were split and after 3 days colonies were infected once. The majority of cells differentiated leaving some colonies with typical ES morphology which were picked mechanically and transferred to human fibroblast feeders. Example of iPS cell colony 7 days (c) and 10 days (d) after picking, demonstrating the typical rapid ES growth rate. e) AP positive hair iPS cell colonies.

FIG. 7 shows: a) iPS cell colony with a cluster of differentiated keratinocytes on top; b) typical iPS cell colony; c) non-iPS cell colony growing in ES medium with typical keratinocyte cell morphology. d) Similar cell colonies only infected with c-Myc; e) GFP infected cells forming small differentiated colonies; Small iPS colonies at 8 days post infection (f), 10 days (g) and 12 days (h); i) Morphology of KiPS3F1 cells adapted to matrigel and showing uniform AP activity (j).

FIG. 8 shows a FACS analysis of KiPS3F1 cells, ES4 cells and human fibroblasts.

FIG. 9 shows a southern blotting analysis to confirm number of retroviral integrations. 1) Endogenous bands: 5.9 kb and 0.9 kb (black arrowheads), additional bands in the different KiPS clones correspond each to single transgene insertions (asterisks); 2) endogenous band: 0.9 kb (black arrowhead) and additional bands in the different KiPS clones correspond each to single transgene insertions (asterisks); 3) endogenous specific band: 4.5 kb (black arrowhead), endogenous unspecific bands (grey arrowheads) and additional bands in the different KiPS clones correspond each to single transgene insertions (asterisks); 4) endogenous band: 11 kb (black arrowhead), additional bands in the different KiPS clones correspond each to single transgene insertions (asterisks); 5) recapitulation of the number of detected transgenes for each KiPS line. N.d. not detected.

FIG. 10 shows a graph depicting total levels of Oct4, Sox2, Klf4, and c-Myc (transgenic and endogenous), as well as of the pluripotency-associated markers Nanog, Cripto, and Rex1, and of a number of markers associated with lineage differentiation, as determined by quatitative RT-PCR on samples of fibroblasts, keratinocytes, human ES cells (ES[4]), and different lines of KiPS cells. These analyses confirm the activation of pluripotency markers and the absence of differentiation in KiPS cells. These analyses also uncover that keratinocytes display much higher expression of Klf4 and c-Myc than fibroblasts.

FIG. 11 shows: a) the expression of Keratin 14 (K14) in primary keratinocytes and its absent in ES4 and KiPS cell lines; b) monitorization of KiPS colony formation shows early reduction of keratin 14 expression in the middle of colonies even at 8 days post infection whereas most cells are K14 negative at day 14 with AP activity found in a mosaic pattern.

FIG. 12 presents the characteristics of hair derived KiPS cells.

### DETAILED DESCRIPTION OF THE INVENTION

The invention relates to the efficient reprogramming of human keratinocytes to pluripotency by retroviral transduction with genes encoding Oct4, Sox2, Klf4 and Myc. Overall, the keratinocyte reprogramming process is, at least, 100-fold more efficient and 2-fold faster than that of fibroblasts. Furthermore the increase in reprogramming efficiency achieved with the system described herein allows the practicability of the iPS technology to be extended, for example, allowing KiPS cells to be generated from a single plucked hair from adult individuals. These developments provide both a valuable experimental model for investigating the basis of cellular reprogramming and make this technology better available for patient treatment.

The system of induced reprogramming of keratinocytes to pluripotency provides a valuable experimental model for investigating the basis of cellular reprogramming and pluripotency, as well as a practically advantageous alternative for the generation of patient- and disease-specific pluripotent stem cells.

### DEFINITIONS

As used herein, the following terms and phrases shall have the meanings set forth below. Unless defined otherwise, all technical and scientific terms used herein have the same meaning as commonly understood to one of ordinary skill in the art to which this invention belongs.

The articles “a” and “an” refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “cellular composition” refers to a preparation of cells, which preparation may include, in addition to the cells, non-cellular components such as cell culture media, e.g. proteins, amino acids, nucleic acids, nucleotides, co-enzyme, anti-oxidants, metals and the like. Furthermore, the cellular composition can have components which do not affect the growth or viability of the cellular component, but which are used to provide the cells in a particular format, e.g., as polymeric matrix for encapsulation or as a pharmaceutical preparation.

The term “culture” refers to any growth of cells, organisms, multicellular entities, or tissue in a medium. The term “culturing” refers to any method of achieving such growth, and may comprise multiple steps. The term “further culturing” refers to culturing a cell, organism, multicellular entity, or tissue to a certain stage of growth, then using another culturing method to bring the cell, organism, multicellular entity, or tissue to another stage of growth. A “cell culture” refers to a growth of cells in vitro. In such a culture, the cells proliferate, but they may not organize into a tissue per se. A “tissue culture” refers to the maintenance or growth of tissue, e.g., explants of organ primordial or of an adult organ in vitro so as to preserve its architecture and function. A “monolayer culture” refers to a culture in which cells multiply in a suitable medium while being principally attached to each other and to a substrate. Furthermore, a “suspension culture” refers to a culture in which cells multiply while suspended in a suitable medium. Likewise, a “continuous flow culture” refers to the cultivation of cells or explants in a continuous flow of fresh medium to maintain cell growth, e.g. viability.

The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells. In other words, a nutrient rich liquid prepared for bacterial culture is a medium. Similarly, a powder mixture that when mixed with water or other liquid becomes suitable for cell culture may be termed a “powdered medium”. “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise four basic chemical groups: amino acids, carbohydrates, inorganic salts, and vitamins. A basal medium generally serves as the basis for a more complex medium, to which supplements such as serum, buffers, growth factors, lipids, and the like are added. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, Mc Coy's 5A, Dulbecco's MEM/F-I 2, RPMI 1640, and Iscove's Modified Dulbecco's Medium (IMDM).

“Dedifferentiation” refers to the loss of characteristics of a specialized cell, and its regression into an undifferentiated or less differentiated state. The dedifferentiated cell may become redifferentiated into a cell of the same cell type as before the dedifferentiation, or into a cell of a different type.

The term “differentiation” refers to the formation of cells expressing markers known to be associated with cells that are more specialized and closer to becoming terminally differentiated cells that are incapable of further division or differentiation. For example, in a pancreatic context, differentiation might be seen as the production of islet-like cell clusters containing an increased proportion of beta epithelial cells that produce increased amounts of insulin. The terms “further” or “greater” differentiation refers to cells that are more specialized and closer to becoming terminally differentiated cells incapable of further division or differentiation than the cells from which they were cultured. The term “final differentiation” refers to cells that have become terminally differentiated cells incapable of further division or differentiation.

The term “transfection” is referred to the introduction of a polynucleotide or nucleic acid into a cell or cell population, which may occur in vivo as well as in vitro. The result of the transfection is the production of genetically engineered cells (transfected cells)). The term “transfection” or “transfecting” also refers to a process of introducing nucleic acid molecules to a cell by non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well-known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well-known in the art.

An “embryonic stem cell” (ES) is a pluripotent cell isolated from a very early embryo. These cells are not differentiated and have the capacity to differentiate into endoderm, ectoderm and endoderm, and further to differentiate into any of the cells in the body. The embryonic stem cell is generally isolated from a very early mammalian embryo, such as a human embryo.

The term “expressed” is used to describe the presence of a marker within a cell. In advantageous embodiments, a marker is considered as being expressed if it is present at a detectable level. By “detectable level” is meant that the marker can be detected using one of the standard laboratory methodologies such as PCR, blotting or FACS analysis. A gene is considered to be expressed by a cell of the population of the invention if expression can be reasonably detected after 30 PCR cycles, which corresponds to an expression level in the cell of at least about 100 copies per cell. The terms “express” and “expression” has corresponding meanings. At an expression level below this threshold, a marker is considered not to be expressed. The comparison between the expression level of a marker in a stem cell of the invention, and the expression level of the same marker in another cell, such as for example an embryonic stem cell, may preferably be conducted by comparing the two cell types that have been isolated from the same species. Preferably this species is a mammal, and more preferably this species is human. Such comparison may conveniently be conducted using a reverse transcriptase polymerase chain reaction (RT-PCR) experiment.

“Fluorescence activated cell sorting (FACS)” is a method of cell purification based on the use of fluorescent labelled antibodies. The antibodies are directed to a marker on the cell surface, and therefore bind to the cells of interest. The cells are then separated based upon the fluorescent emission peak of the cells.

The term “including” is used herein to mean “including but not limited to”. “Including” and “including but not limited to” are used interchangeably.

The term “isolated” indicates that the cell or cell population to which it refers is not within its natural environment. The cell or cell population has been substantially separated from surrounding tissue. In some embodiments, the cell or cell population is substantially separated from surrounding tissue if the sample contains at least about 75%, in some embodiments at least about 85%, in some embodiments at least about 90%, and in some embodiments at least about 95% target cells. In other words, the sample is substantially separated from the surrounding tissue if the sample contains less than about 25%, in some embodiments less than about 15%, and in some embodiments less than about 5% of materials other than the target cells. Such percentage values refer to percentage by weight. The term encompasses cells which have been removed from the organism from which they originated, and exist in culture. The term also encompasses cells which have been removed from the organism from which they originated, and subsequently re-inserted into an organism. The organism which contains the re-inserted cells may be the same organism from which the cells were removed, or it may be a different organism.

“Marker” or “factor” refers to a biological molecule whose presence, concentration, activity, or phosphorylation state may be detected and used to identify the phenotype of a cell.

The term “passage” refers to a method of sub-culturing cells. Passaging is advantageous when a large number of cells are being grown, and without it the cells would exhaust the nutrient supply of the media, become compressed against each other and die. Generally, cells are grown in a flask or dish with a supply of nutrient media, where they adhere to the bottom of the dish or to a layer of feeder cells (mitotically-inactivated primary human or mouse fibroblasts), and can become confluent in 5-7 days. In order to passage the cells, the media is removed and the cells are generally washed before being treated with trypsin to reduce their adherence to the surface on which they are grown, or small fragments of cell colonies are picked mechanically. The cells or colony fragments are then suspended in culture media before an appropriate number of cells are transferred to a new dish.

The term “progenitor cell” refers to a cell that has the capacity to create progeny that are more differentiated than itself. For example, the term may refer to an undifferentiated cell or cell differentiated to an extent short of final differentiation, which is capable of proliferation and giving rise to more progenitor cells having the ability to generate a large number of mother cells that can in turn give rise to differentiated or differentiable daughter cells. In one embodiment, the term progenitor cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. By this definition, stem cells may also be progenitor cells, as well as the more immediate precursors to terminally differentiated cells.

“Proliferation” refers to an increase in cell number. “Proliferating” and “proliferation” refer to cells undergoing mitosis.

The term “pluripotent” refers to cells which are capable of differentiating into cell derivatives of the three embryo germ lineages (endoderm, ectoderm, and mesoderm).

In the context of this application the term “tripotent” refers to a cell which, although it may not be pluripotent, is capable of generating cell types corresponding to the three layers of the early embryo; mesoderm, endoderm and ectoderm.

The term “substantially pure” as used herein, refers to a population of stem cells that is at least about 75%, in some embodiments at least about 85%, in some embodiments at least about 90%, and in some embodiments at least about 95% pure, with respect to other cells that make up a total cell population. For example, with respect to cardiac tissue-derived stem cell populations, this term means that there are at least about 75%, in some embodiments at least about 85%, in some embodiments at least about 90%, and in some embodiments at least about 95% pure, cardiac stem cells compared to other cells that make up a total cell population. In other words, the term “substantially pure” refers to a population of stem cells of the present invention that contain fewer than about 25%, in some embodiments fewer than about 15%, and in some embodiments fewer than about 5%, of lineage committed cells in the original unamplified and isolated population prior to subsequent culturing and amplification.

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

“Tissue regeneration” is the process of increasing the number of cells in a tissue following a trauma. The trauma can be anything which causes the cell number to diminish. For example, an accident, an autoimmune disorder or a disease state could constitute trauma. Tissue regeneration increases the cell number within the tissue and enables connections between cells.

A “patient”, “subject” or “host” to be treated by the subject method may mean either a human or non-human animal.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Advantageously, each carrier is “acceptable” in the sense of being compatible with the other ingredients of the formulation and not injurious to the patient.

### Cells of the Invention

An aspect of the present invention relates to a new type of iPS cells, KiPS cells, which are derived from transfected keratinocytes, preferably human keratinocytes and more preferably primary human keratinocytes, which are also part of the invention (here and after, referred to as transfected keratinocytes). So, in a first aspect, the present invention provides a keratinocyte or keratinocytes population which has been transfected with the genes selected from the group comprising: an Oct4 family gene; a Sox2 family gene and a Klf family gene. In one embodiment of the invention the group of genes also comprises a Myc family gene, more preferably the c-Myc gene and even more preferably c-Myc^{T58A} The transfection of these genes confers to the keratinocytes the capacity to dedifferentiate into ES-like stem cells.

In another embodiment of the invention, one or more of the genes can comprise a promoter to modulate its expression. Furthermore, it is preferred that one or more of the genes is cloned in a vector, which facilitates its transfection and/or its expression. In one embodiment of the invention this vector is a viral vector (adenovirus, retrovirus, adeno-associated virus, or other vector), preferably, selected from the group which comprises, without any kind of limitation, murine stem cell virus (MSCV) derived retroviral vectors or Moloney murine leukemia virus (MMLV) based vectors. In certain embodiments, when the murine stem cell virus (MSCV) derived retroviral vectors is used, the cells to be transfected can be any somatic cell (transfected somatic cell). When placed in an appropriate media, these transfected somatic cells have the ability to dedifferentiate into ES-like somatic cells, which are also part of the present invention.

Another aspect of the present invention comprises a population of transfected keratinocytes as a substantially pure population. In a particular aspect, the invention comprises a cell population which comprises at least about 80% (in other aspects at least about 85%, 90%, 91%, 92%, 93%, 94%, 95%, 96%, 97%, 98%, 99% or 100%) transfected keratinocytes of the invention.

The transfected keratinocytes can be induced to dedifferenciate into one or more KiPS cells upon addition of the appropriate medium which facilitates or promotes its dedifferentiation. In one embodiment this media is an ES cell media or hES cell media. Thus, another aspect of the present invention provides a KiPS cell or a KiPS population. These KiPS cells or KiPS populations are characterized in that the cells have pluripotent capacity and, in certain aspects, tripotent capacity or potential. As defined above, this tripotent potential allows the cells to develop into cells derived from the endoderm, mesoderm and ectoderm. In certain aspects, the KiPS populations or KiPS cells are pluripotent if the cells are capable of differentiating into at least one cell type of each of: an endodermal cell type, an ectodermal cell type and a mesodermal cell type. In certain embodiments, the KiPS populations are considered to have pluripotent or tripotent potential if at least about 70% of the cell population show, either pluripotent or tripotent capacity. In other embodiments, at least about 80%, (in other aspects at least about 85%, 90%, 91%, 92%, 93%, 94%, 95%, 96%, 97%, 98%, 99% or 100%) of the cells of the KiPS population show, either, pluripotent or tripotent capacity potential. Tripotent potential can be determined by forming the cells into embryoid bodies and culturing the embryoid bodies in specific differentiation media. The cells can then be amplified and differentiation confirmed by quantitative PCR, using lineage-restricted transcripts.

Furthermore, another characteristic of the KiPS cells of the invention is that they are able to remain in culture as undifferentiated cells through a number of passages, and do not differentiate until they are provided with appropriate differentiating media. Upon administration of appropriate differentiating media, the KiPS cells are capable of differentiating into any cell or tissue with of mesodermal, ectodermal or endodermal origin.

### Cell Markers

As indicated above, the invention provides an isolated population of cells with pluripotent and/or tripotent capacity or potential (KiPS cells population), which are derived from the transfected keratinocytes of the invention.

The KiPS population of the invention is considered to express a marker if at least about 70% of the cells of the population show detectable expression of the marker. In other aspects, at least about 80%, at least about 90% or at least about 95% or at least about 97% or at least about 98% or more of the cells of the population show detectable expression of the marker. In certain aspects, at least about 99% or 100% of the cells of the population show detectable expression of the markers. Expression may be detected through the use of an RT-PCR experiment or through fluorescence activated cell sorting (FACS). It should be appreciated that these examples of suitable methodologies for determining expression is provided by way of example only, and is not intended to be limiting.

The markers described below are considered to be expressed by a cell of the population of the invention, if expression can be reasonably detected after 30 PCR cycles, which corresponds to an expression level in the cell of at least about 100 copies per cell.

In one of the primary aspects of the invention, the KiPS cells of the invention are characterized by the expression of one or more of the transcription factors or surface markers selected from the group which comprises: Nanog, Oct 4, Sox2, Rex1, Cripto, Connexin43, IGF-1 receptor, SSEA4, SSEA3, Tra-1-61 and Tra-1-81. In yet another preferred aspect the KiPS cells of the invention also express one or more of the following markers: AP marker, CD24, CD24, CD90, CD29, CD9 and CD49f.

In certain embodiments of the invention, the KiPS cells express one or more of the markers Rex1, Cripto and AP. In yet another preferred embodiment the KiPS cells also express one or more of the markers selected from the group which comprises IGF1R, CD24, CD90, CD29, CD9 and CD49f but do not express Keratin 14.

In yet another aspect of the present invention, the population of KiPS cells is a substantially pure population. In a particular aspect, the invention comprises a cell population which comprises at least about 80% (in other aspects at least about 85%, 90%, 91%, 92%, 93%, 94%, 95%, 96%, 97%, 98%, 99% or 100%) KiPS cells of the invention.

### Method of Obtaining KiPS Cells and Other iPS Cells

Another aspect of the present invention provides a method for obtaining KiPS cells which comprises the transfection of a polynucleotide or polynucleotides into an isolated keratinocyte or isolated keratinocyte population, said polynucleotide or polynucleotides encoding the following group of genes: an Oct4 family gene; Sox2 family gene, a Klf family gene. In a preferred aspect the group of genes also comprises a c-Myc family gene, preferably c-Myc^{T58A}. Preferably, the isolated keratinocyte or keratinocytes population is obtained by plating a hair or a root hair in a culture media which promotes the keratinocytes proliferation.

In a further aspect of the method of the invention, the polynucleotide or polynucleotides also comprise a promoter to regulate the expression of the genes. Preferably, the polynucleotide or polynucleotides are cloned in a vector, preferably the Moleney murine leukemia virus (MMLV) based vector and, more preferably, a murine stem cell virus (MSCV) derived retroviral vector.

In yet another further aspect, the invention also provides a method for obtaining obtaining iPS cells, which comprises the previous selection of the adult somatic cells to be transfected. This selection is based on the finding that the high endogenous expression of the genes c-Myc or Klf4 provides somatic cells which can be reprogrammed into iPS easily. Thus, the method for obtaining iPS cells comprises the following steps:
a. Positively selecting an isolated somatic cell or an isolated somatic cell population by the comparison of the expression level of the markers c-Myc or Klf4 with an isolated fibroblast cell or an isolated fibroblast population, wherein said positive selection is made where the expression level of either of said markers is at least 10, at least 15 or at least 30 fold higher in comparison with the expression level in the isolated fibroblast cell or the isolated fibroblast population,
b. Transfecting the selected isolated somatic cell or the selected isolated somatic population selected in step a) with a polynucleotide or polynucleotides encoding the following group of genes: an Oct4 family gene; Sox2 family gene, a Klf family gene and optionally c-Myc, and
c. Placing the transfected isolated somatic cell or isolated cell population in an appropriate dedifferentiating medium.

In one embodiment the expression level of c-Myc or Klf4 in the isolated somatic cells or isolated somatic cell population is, between at least about 14 and about 30 fold higher.

In one embodiment of this method, the polynucleotide or polynucleotides also comprises a promoter to regulate the expression of the genes. Preferably, the polynucleotide or polynucleotides are cloned in a vector, preferably the Moleney murine leukemia virus (MMLV) based vector and, more preferably, a murine stem cell virus (MSCV) derived retroviral vector.

### Pharmaceutical Composition.

As is demonstrated herein, the transfected keratinocytes and the KiPS cells of the invention are able to differentiate into cells of different embryonic germ layer origins (ectoderm, mesoderm and endoderm). Consequently, another aspect of the present invention relates to a pharmaceutical composition, which comprises transfected keratinocytes, KiPS cells and/or the progeny thereof, for use as a medicament, preferably, for use in the regeneration of a tissue with mesenchymal, ectodermal or endodermal origin.

The pharmaceutical composition of the invention may include a substantially pure population of transfected keratinocytes, the KiPS population of the invention and/or the progeny thereof. The composition of the present invention may also include cell culture components, e.g., culture media including one or more of amino acids, metals and coenzyme factors. The composition may also include other non-cellular components which may support the growth and survival of the cells of the invention or the progeny thereof under particular circumstances, e.g. implantation, growth in continuous culture, or use as a biomaterial or composition.

The pharmaceutical composition of the invention may comprise a population of cells in which at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, of the cells of the composition are the transfected keratinocytes and/or the KiPS population of the invention or the progeny thereof. In other words, in some embodiments at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, of the cells in the composition are the transfected keratinocytes and/or the KiPS cells or the progeny thereof.

The pharmaceutical composition of the invention may comprise at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%, of the transfected keratinocytes and/or the KiPS cells and/or the progeny thereof, either calculated by number, or by weight or by volume of the composition. In one embodiment the pharmaceutical composition of the invention comprises at least about 80% KiPS cells.

The concentration of the transfected keratinocytes and/or the KiPS cells and/or the progeny thereof in the pharmaceutical composition of the invention may be at least about 1×10⁴ cells/mL, at least about 1×10⁵ cells/mL, at least about 1×10⁶ cells/mL, at least about 10×10⁶ cells/mL, or at least about 40×10⁶ cells/mL. In one embodiment the pharmaceutical composition has a concentration of at least about 1×10⁶ KiPS cells.

In certain embodiments, the transfected keratinocytes and/or the KiPS cells and/or the progeny thereof of the pharmaceutical composition of the invention are provided sterile, free of the presence of unwanted virus, bacteria and other pathogens, as well as a pyrogen-free preparation. That is, for human administration, the subject compositions should meet sterility, pyrogenicity as well as general safety and purity standards as required by FDA and EMEA.

In certain embodiments, the pharmaceutical composition of the invention is prepared to be systemically or locally administered, preferably by parenteral route (intravenous route, intramuscular route, intradermic route, subdermic route or intrabone route) into animals, preferably mammals, and even more preferably humans. The cells can be preferably autologous, but also allogeneic or xenogeneic with respect to the transplantation host.

Methods of administering a pharmaceutical composition of the invention to subjects, particularly human subjects, which are described in detail herein, include injection or implantation of the cells into target sites in the subjects, the cells can be inserted into a delivery device which facilitates introduction by, injection or implantation, of the cells into the subjects. Such delivery devices include tubes, e.g., catheters, for injecting cells and fluids into the body of a recipient subject. In one embodiment, the tubes additionally have a needle, e.g., a syringe, through which the pharmaceutical composition of the invention can be introduced into the subject at a desired location for a local or systemic administration. The cellular component of the pharmaceutical composition can be inserted into such a delivery device, e.g., a syringe, in different forms.

Pharmaceutically acceptable carriers and diluents include saline, aqueous buffer solutions, solvents and/or dispersion media. The use of such carriers and diluents is well known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. The pharmaceutical compositions of the invention can be prepared by incorporating the transfected keratinocytes and/or the KiPS cells and/or the progeny thereof in a pharmaceutically acceptable carrier or diluent and, as may be advantageously used, other ingredients enumerated above, followed by filter sterilization.

Some examples of materials and solutions which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Provided, herein, are also methods for preparing the pharmaceutical composition of the invention containing therapeutic agents. For example, the pharmaceutical composition may contain an analgesic or an antibiotic, preferably a broad-spectrum antibiotic.

More specifically, non-limiting examples of useful therapeutic agents include the following therapeutic categories: analgesics, such as nonsteroidal anti-inflammatory drugs, opiate agonists and salicylates; anti-infective agents, such as antihelmintics, antianaerobics, antibiotics, aminoglycoside antibiotics, antifungal antibiotics, cephalosporin antibiotics, macrolide antibiotics, miscellaneous β-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, anti-inflammatory agents, corticosteroid anti-inflammatory agents, antipruritics/local anesthetics, topical anti-infectives, antifungal topical anti-infectives, antiviral topical anti-infectives; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hormones and hormone modifiers, such as abortifacients, adrenal agents, corticosteroid adrenal agents, androgens, anti-androgens, immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs (NSAIDs), salicylate anti-inflammatory agents, minerals; and vitamins, such as vitamin A, vitamin B, vitamin C, vitamin D, vitamin E, and vitamin K.

Preferred classes of useful therapeutic agents from the above categories include: (1) analgesics in general, such as lidocaine or derivatives thereof, and nonsteroidal anti-inflammatory drugs (NSAIDs) analgesics, including diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) H1-blocker antihistamines, such as clemastine and terfenadine; (5) anti-infective agents, such as mupirocin; (6) antianaerobic anti-infectives, such as chloramphenicol and clindamycin; (7) antifungal antibiotic anti-infectives, such as amphotericin b, clotrimazole, fluconazole, and ketoconazole; (8) macrolide antibiotic anti-infectives, such as azithromycin and erythromycin; (9) miscellaneous β-lactam antibiotic anti-infectives, such as aztreonam and imipenem; (10) penicillin antibiotic anti-infectives, such as nafcillin, oxacillin, penicillin G, and penicillin V; (11) quinolone antibiotic anti-infectives, such as ciprofloxacin and norfloxacin; (12) tetracycline antibiotic anti-infectives, such as doxycycline, minocycline, and tetracycline; (13) antituberculosis antimycobacterial anti-infectives such as isoniazid (INH), and rifampin; (14) antiprotozoal anti-infectives, such as atovaquone and dapsone; (15) antimalarial antiprotozoal anti-infectives, such as chloroquine and pyrimethamine; (16) anti-retroviral anti-infectives, such as ritonavir and zidovudine; (17) antiviral anti-infective agents, such as acyclovir, ganciclovir, interferon alfa, and rimantadine; (18) antifungal topical anti-infectives, such as amphotericin B, clotrimazole, miconazole, and nystatin; (19) antiviral topical anti-infectives, such as acyclovir; (20) electrolytic and renal agents, such as lactulose; (21) loop diuretics, such as furosemide; (22) potassium-sparing diuretics, such as triamterene; (23) thiazide diuretics, such as hydrochlorothiazide (HCTZ); (24) uricosuric agents, such as probenecid; (25) enzymes such as RNase and DNase; (26) antiemetics, such as prochlorperazine; (27) salicylate gastrointestinal anti-inflammatory agents, such as sulfasalazine; (28) gastric acid-pump inhibitor anti-ulcer agents, such as omeprazole; (29) H2-blocker anti-ulcer agents, such as cimetidine, famotidine, nizatidine, and ranitidine; (30) digestants, such as pancrelipase; (31) prokinetic agents, such as erythromycin; (32) ester local anesthetics, such as benzocaine and procaine; (33) musculoskeletal corticosteroid anti-inflammatory agents, such as beclomethasone, betamethasone, cortisone, dexamethasone, hydrocortisone, and prednisone; (34) musculoskeletal anti-inflammatory immunosuppressives, such as azathioprine, cyclophosphamide, and methotrexate; (35) musculoskeletal nonsteroidal anti-inflammatory drugs (NSAIDs), such as diclofenac, ibuprofen, ketoprofen, ketorlac, and naproxen; (36) minerals, such as iron, calcium, and magnesium; (37) vitamin B compounds, such as cyanocobalamin (vitamin B12) and niacin (vitamin B3); (38) vitamin C compounds, such as ascorbic acid; and (39) vitamin D compounds, such as calcitriol.

In certain embodiments, the therapeutic agent may be a growth factor or other molecule that affects cell proliferation or activation. Growth factors that induce final differentiation states are well-known in the art, and may be selected from any such factor that has been shown to induce a final differentiation state. Growth factors for use in methods described herein may, in certain embodiments, be variants or fragments of a naturally-occurring growth factor. For example, a variant may be generated by making conservative amino acid changes and testing the resulting variant in one of the functional assays described above or another functional assay known in the art. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

As those skilled in the art will appreciate, variants or fragments of polypeptide growth factors can be generated using conventional techniques, such as mutagenesis, including creating discrete point mutation(s), or by truncation. For instance, mutation can give rise to variants which retain substantially the same, or merely a subset, of the biological activity of a polypeptide growth factor from which it was derived.

### EXAMPLES

The invention now being generally described, will be more readily understood by reference to the following examples, which are included merely for purposes of illustration of certain aspects and embodiments of the present invention, and are not intended to limit the invention.

### Example 1

### Production of KiPS

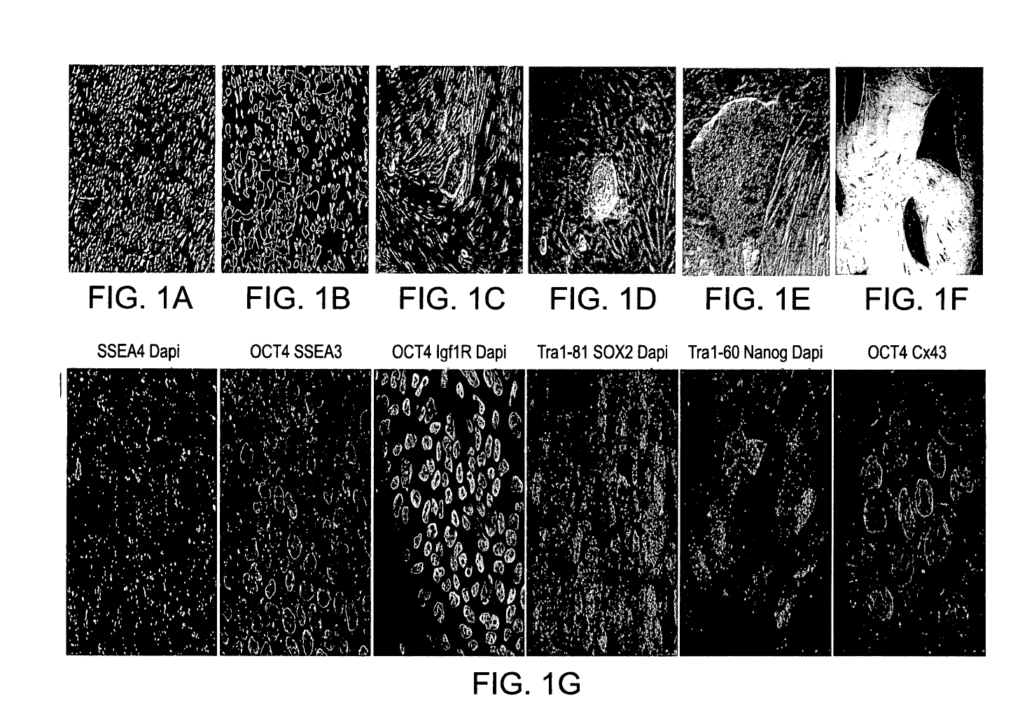

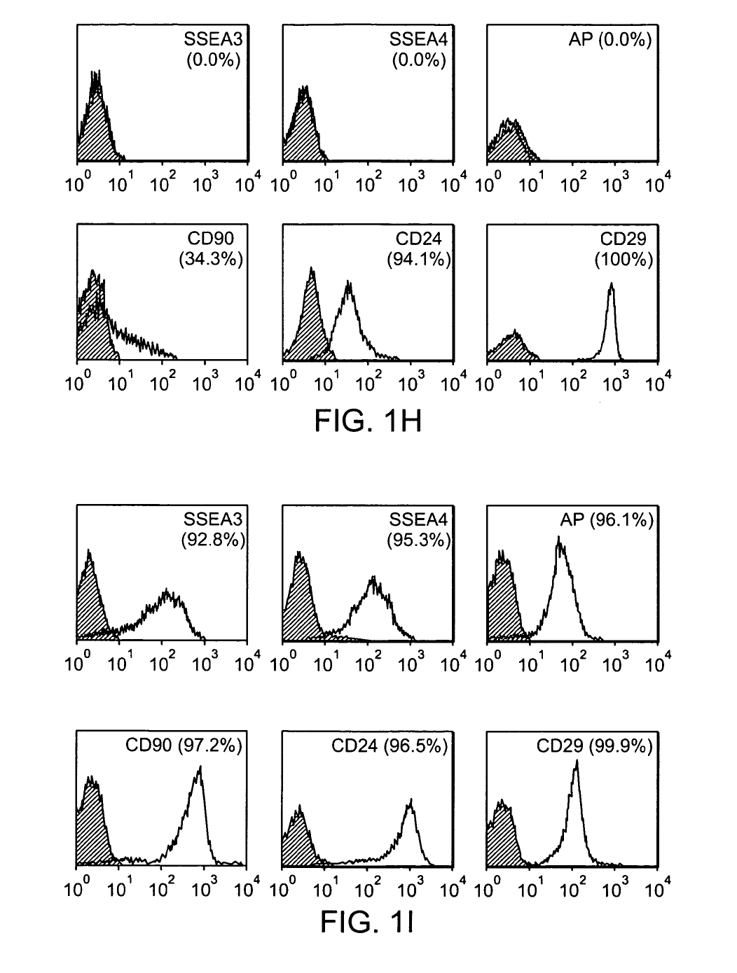

Epidermal keratinocytes were obtained from a biopsy of normal human foreskin (age 4 years) and cultured in serum free and low-calcium medium, which facilitates a highly proliferative undifferentiated state. N-terminal FLAG-tagged versions of human Oct4, Sox2, Klf4, and c-Myc, or GFP were cloned into a murine stem cell virus-(MSCV) derived retroviral vector, which provides higher transcriptional activation than commonly-used Moloney murine leukemia virus (MMLV) based vectors (Hawley, R. G et al. (1994) Gene therapy 1, 136-138). Primarily, retroviral transduction of keratinocytes was optimized with GFP and it was found that two 45-min spinfections at 750 g 24 h apart resulted in nearly 100% infection of undifferentiated cells (FIGS. 1 a-b). This infection protocol was then used to transduce keratinocytes with retroviruses encoding Oct4, Sox2, Klf4, and c-Myc. Fifty thousand passage 1-5 keratinocytes were seeded (day 0) and infected on days 1 and 2 with a 1:1:1:1 mixture of retroviruses. Cells were trypsinized on day 4 and seeded onto a layer of irradiated mouse embryonic fibroblasts (MEFs) in ES cell medium. Within 2-3 days (6-7 days post-infection), several hundred small, tight cell colonies that grew rapidly were detected and, by day 10 post-infection, these colonies displayed typical hES cell like morphology (tight colonies of cells with large nuclei to cytoplasmic ratio and prominent nucleoli; FIG. 1c). Further, clumps of differentiated non-proliferating cells (FIG. 1d and FIG. 7a) and a few colonies with typical keratinocyte morphology were observed, although both of these were easily distinguishable from hES cell-like colonies (FIGS. 7b,c). Similar results were obtained with keratinocytes isolated from two other independent normal foreskin biopsies of 4- and 16-year old patients. Transduction of keratinocytes with single factors or GFP did not result in the formation of cell colonies, except for c-Myc, in which large numbers of colonies with distinct keratinocyte morphology were observed (FIG. 7d-e, and data not shown). Thus, as shown in this Example, hES cell-like colonies were obtained with the combination of: Oct4, Sox2, Klf4 and, optionally, c-Myc.

### Materials and Methods

### Cell Culture

Keratinocytes were isolated from juvenile foreskins (4-16 year old) using dispase to remove the dermis from the epidermis followed by trypsinization of the epidermis and culture in serum free low calcium medium (Epilife, Invitrogen). A 1:1:1:1 mix of retroviruses with FLAG-tagged Oct4, Sox2, Klf4, and c-Myc^{T58A} was added to keratinocytes (between passage 1 and 5) in the presence of 1 ug/ml polybrene and spinfected for 45 minutes at 750 g. After replacing with fresh serum free low calcium medium and incubating for 2 days cells were trypsinised and seeded into 10 cm dishes containing 4 million irradiated mouse fibroblasts and ES medium. ES cells and KiPS cells were cultured either on top of irradiated mouse or human fibroblasts and picked mechanically, or on matrigel by trypzinization (using mouse fibroblast conditioned media). Knockout DMEM ES media was supplemented with 20% knockout serum replacement, non-essential amino acids, 2Mercaptoethanol, Penicillin/Streptomycin, GlutaMAX, bFGF (all Gibco) and Human albumin (Grifols).

### Constructs and Retroviral Production

cDNAs for Oct4 and Sox2 were amplified from ES total RNA by RT-PCR. Klf-4 was amplified from IMAGE clone 5111134. C-Myc T58A mutant cDNA was used. The amplified cDNAs were cloned into the EcoRI/ClaI sites of a modified pMSCVpuro vector (Clontech) that allows the expression of N-terminal FLAG tagged proteins. Retroviruses for the four factors were independently produced after transfecting the cell line Phoenix Amphotropic using Fugene 6 reagent (Roche) according to manufacturer>>s directions. After 24 hours medium was replaced, cells were incubated at 32 Celsius, and viral supernatant was harvested after 24 and 48 hours.

### Example 2

### Characterisation of the KiPS

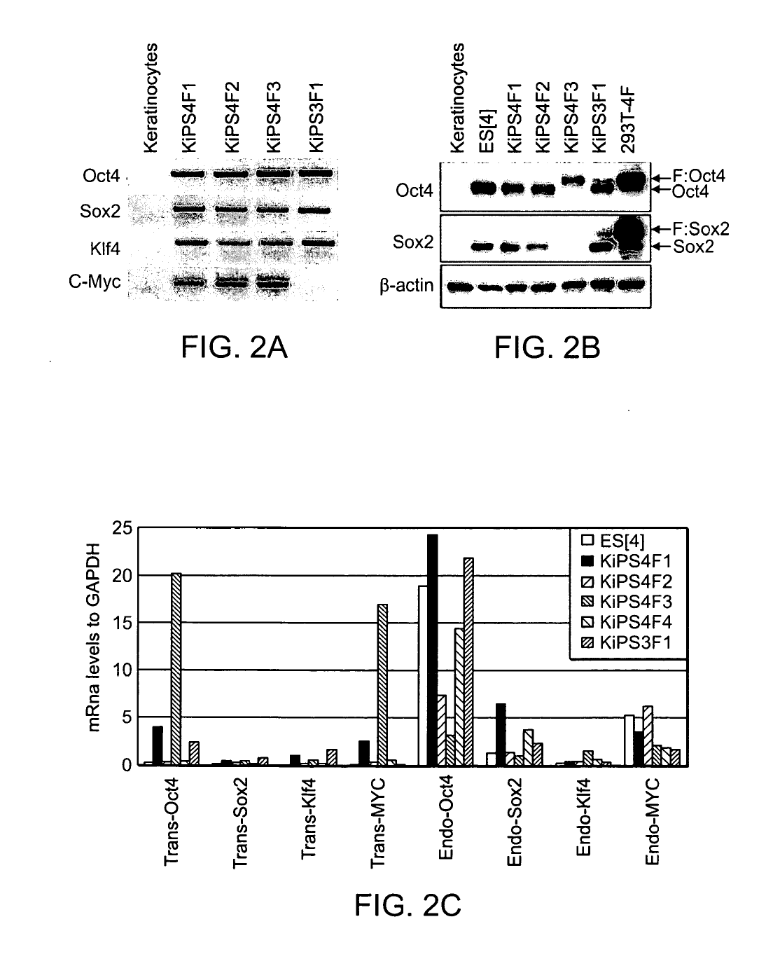

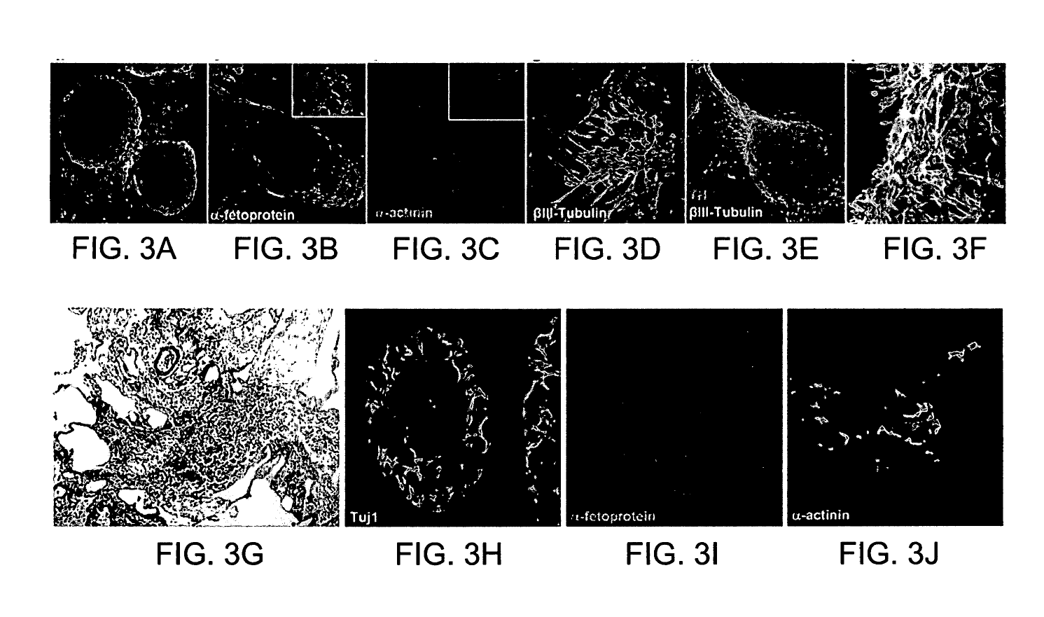

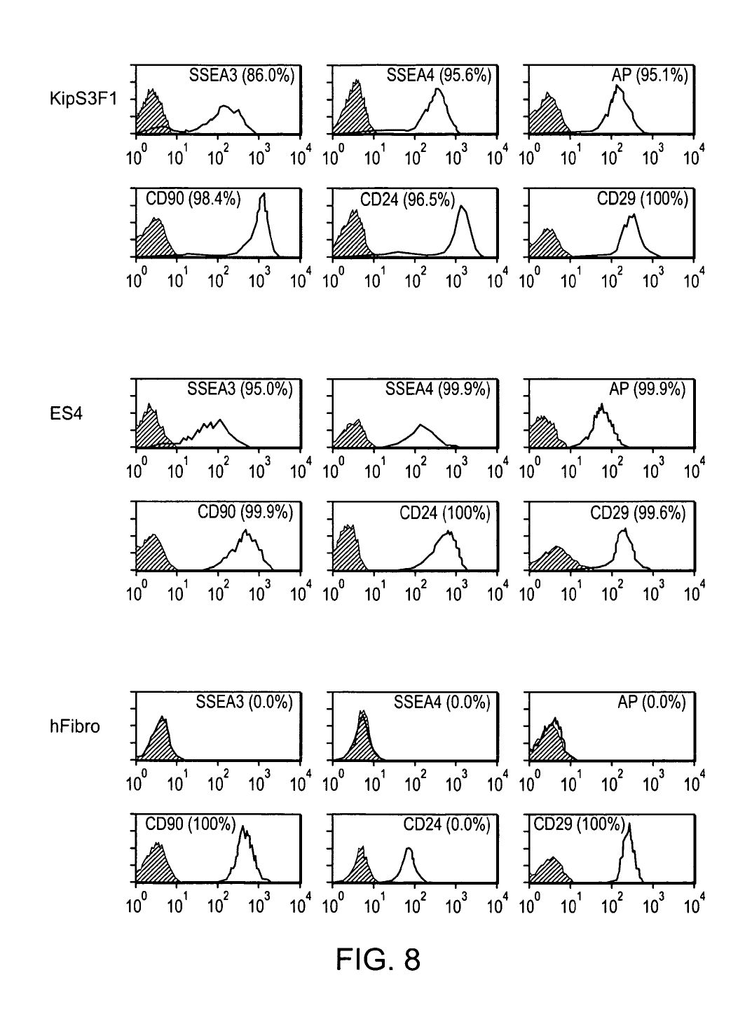

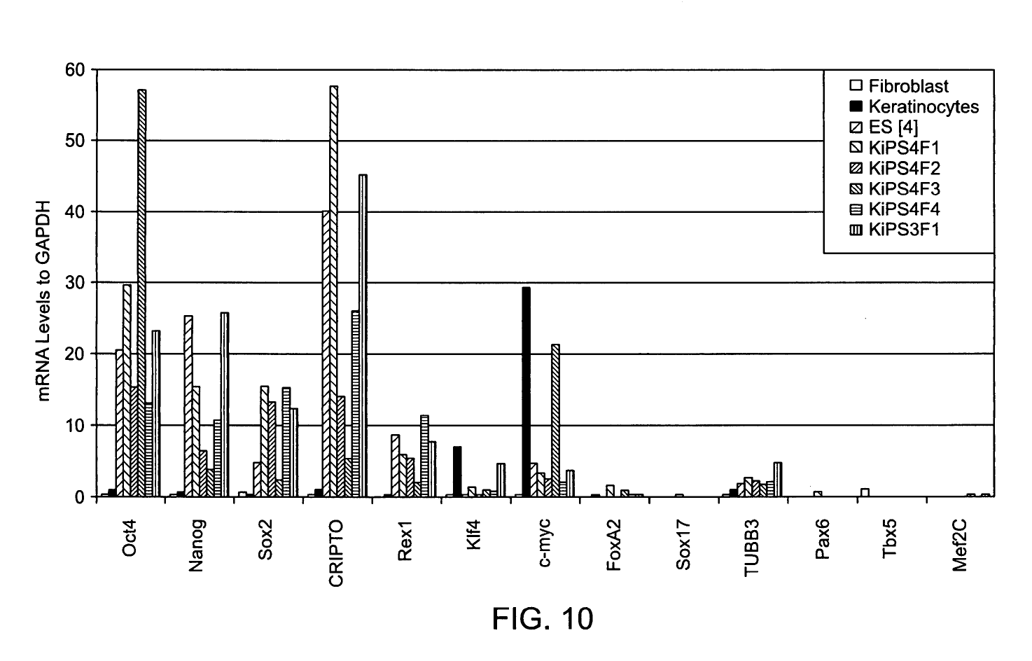

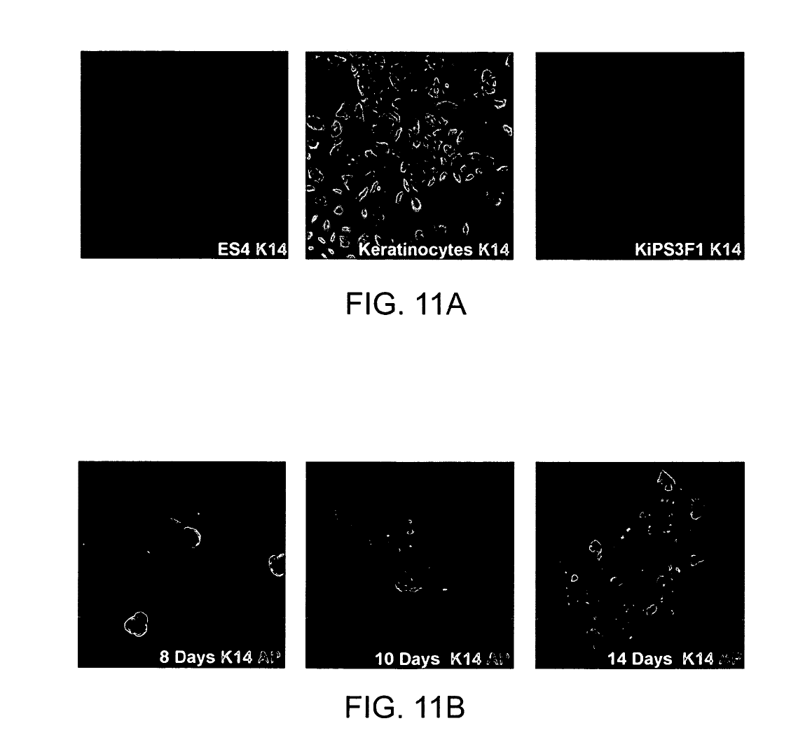

In order to ascertain the nature of the KiPS cells, 24 and 5 colonies obtained after transduction with the 4 factors or without c-Myc, respectively were picked, and passaged as fragments onto fresh feeder cells. Most of these subcultures (21 out of 24 and 5 out of 5) expanded and gave rise to colonies with hES cell-like morphology that stained strongly positive for alkaline phosphatase (AP) activity (FIG. 1f). Six lines were selected for further analyses (which the Inventors putatively termed keratinocyte-derived iPS-KiPS-cells), 4 lines obtained with 4 factors (KiPS4F1-4, of which KiPS4F1 was the result of pooling ˜20 independent colonies) and 2 lines generated without c-Myc (KiPS3F1-2). Except for the KiPS4F3 line, which displayed a strong tendency to spontaneously differentiate in culture (see below), all the other lines could be easily maintained by standard procedures for the culture of hES cells, passaged mechanically onto feeder cells or adapted to enzymatic passaging as single cells on matrigel-coated plates with feeder-conditioned media (FIGS. 7 i-j), displayed a normal 46 XY karyotype (data not shown), and an HLA haplotype identical to that of fibroblasts obtained from the original foreskin biopsy (A*01,24, B*44,44, DRB1*07,13). Some of these lines (KiPS4F1-2 and KiPS3F1) were maintained in continuous culture for over 5 months without signs of replicative crisis. Furthermore, KiPS cells expressed genes and cell surface markers characteristic of hES cells, including Nanog, Oct4, Sox2, Rex1, Cripto, Connexin43, IGF1 receptor, SSEA3, SSEA4, Tra-1-61, and Tra-1-81 (FIG. 1g), as well as complete loss of keratinocyte specific markers such as Keratin 14 (FIG. 10 and data not shown). A detailed flow cytometry analysis was conducted on two lines (KiPS4F1 and KiPS3F1). At passage 6, KiPS4F1 and KiPS3F1 cells were 85-100% positive for all ES cell markers tested, including SSEA3, SSEA4 and AP, as well as IGF1R, CD24, CD90, CD29, CD9 and CD49f (FIG. 1i, FIG. 8, and data not shown). Overall, the expression of stem cell markers in KiPS cells was indistinguishable from that of hES cell lines (Raga A, Rodriguez-Piza I, Aran B, Consiglio A, Barri P N, Veiga A, Izpisua-Belmonte J C. Generation of cardiomyocytes from new human embryonic stem cell lines derived from poor-quality blastocysts. Cold Spring Harbor Symp Quant Biol 73, in press) and maintained under similar conditions (FIGS. 8 and 11, see also FIGS. 2b,c and FIG. 3j). These results indicate that KiPS cells display the main characteristics of hES cells in terms of colony morphology, growth properties, karyotypic stability, expression of pluripotency-associated transcription factors and surface markers.

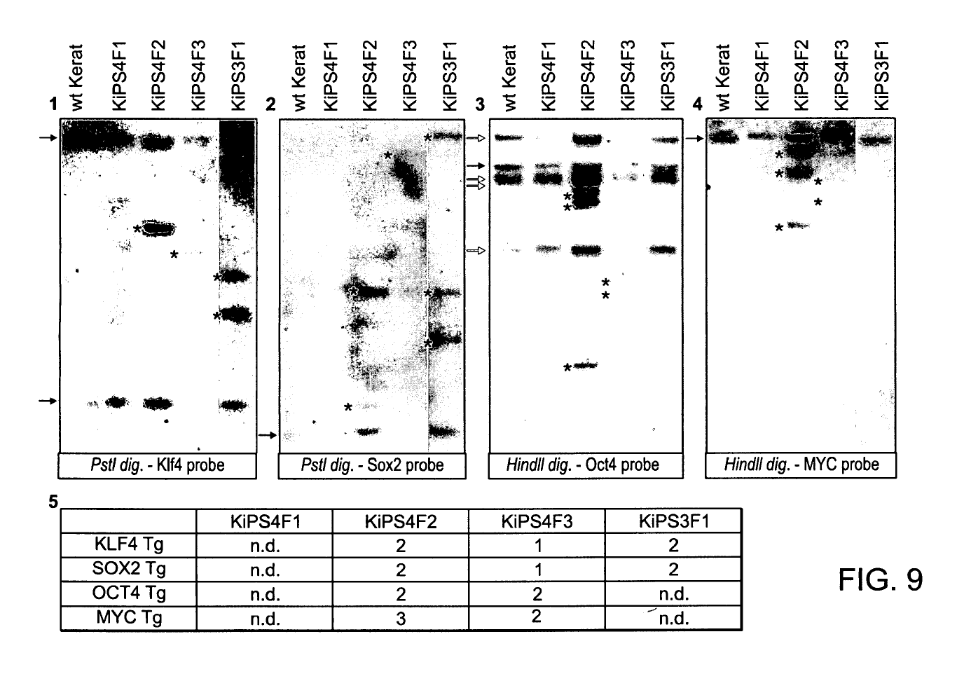

Southern blot analysis of genomic DNA confirmed the independent origin of the KiPS cell lines (FIG. 9). Consistent with the origin of KiPS4F1 as a pool of independent KiPS colonies, the hybridization signal was diluted in Southern blot and no specific bands were detectable, but PCR of genomic DNA clearly demonstrated the integration of all four transcription factors (FIG. 2a). The absence of c-Myc retroviral integrations in KiPS3F1 was confirmed by PCR and Southern blotting of genomic DNA (FIG. 2a and FIG. 9). Interestingly, the number of retroviral integrations in KiPS cells (maximum of 3 and average of 2 integrations per retrovirus and genome) is clearly lower than that of iPS cells generated from human fibroblasts.

To detect the correct generation of KiPS cells, which required the non-expression of Oct4, Sox2, Klf4, or c-Myc, different assays were carried out taking advantage of the fact that exogenous factors expressed in the system were FLAG-tagged. Except for the KiPS4F3 line, slower-migrating, tagged factors in KiPS cell extracts were not detected by Western blot with specific antibodies against Oct4, Sox2, Klf4, or c-Myc, nor was anti-FLAG immunoreactivity detected (FIG. 2b and data not shown). These results were confirmed by quantitative RT-PCR using primers specific for either the endogenous or transgenic factors (FIG. 2c). Interestingly, the incomplete silencing of Oct4 and c-Myc transgenic expression in KiPS4F3 cells was associated with deficient reprogramming, as evidenced by their tendency to spontaneously differentiate and their failure to activate the endogenous expression of Oct4, Sox2, Nanog, Rex1, and Cripto (FIG. 2c and FIG. 10).

### Materials and Methods

### Western Blot Analysis of the Clones

Whole cell extracts were isolated using RIPA buffer and 10 μg protein was analyzed by western blot using specific antibodies against Oct4 (Santa Cruz sc-5279), Sox2 (Neuromics GT15098), c-myc (Sigma C3956), Klf4 (Santa Cruz sc-12538) or FLAG (Sigma M2).

### Immunofluoresence Analysis

Cells were grown on plastic coverslide chambers, fixed with 4% PFA. The following antibodies were used: keratin 14 (Covance, PRB-155P, 1:1000), alkaline phosphatase (Abcam, ab17973-50, 1:50). Tra-1-60 (MAB4360, 1:100), Tra-1-81 (MAB4381, 1:100), Sox2, AB5603, 1:500) all Chemicon, SSEA-4 (MC-813-70, 1:2), SSEA-1 (MC-480, 1:2), SSEA-3 (MC-631, 1:2) all Iowa, Tuj1 (1:500; Covance), TH (1:1000; Sigma), αfetoprotein (1:400; Dako), α-actinin (1:100; Sigma), Oct-3/4 (C-10, SantaCruz, sc5279, 1:100), Nanog (Everest Biotech EB06860, 1:100). Images were taken using Leica SP5 confocal microscope.

### Southern Blotting

Genomic DNA from each cell line was isolated with a standard protocol using Proteinase K and Phenol (Molecular Cloning, Sambrook and Russel, CSHL Press, third edition). Approx. 3 micrograms of each DNA sample were digested with 40 U of PstI or HindIII restriction enzyme (New England Biolabs), electrophoresed on a 1% agarose gel, transferred to neutral nylon membranes (Hybond-N, Amersham, Piscataway, N.J., USA) and hybridised with DIG-dUTP labelled probes generated by PCR using the PCR DIG Probe Synthesis Kit (Roche Diagnostics GmbH, Mannheim, Germany) Probes were detected by an Alkaline Phosphatase conjugated DIG-Antibody (Roche Diagnostics GmbH, Mannheim, Germany) using CDP-Star (Sigma-Aldrich) as a substrate for chemiluminescence. Conditions were per the instructions of the manufacturer. The probes were generated using SOX2, OCT4, KLF4 and MYC cDNAs as templates with the following primers:

SOX2 F
5′ AGTACAACTCCATGACCAGC 3′
SOX2 R
5′ TCACATGTGTGAGAGGGGC 3′
OCT4 F
5′ TAAGCTTCCAAGGCCCTCC 3′
OCT4 R
5′ CTCCTCCGGGTTTTGCTCC 3′
KLF4 F
5′ AATTACCCATCCTTCCTGCC 3′
KLF4 R
5′ TTAAAAATGCCTCTTCATGTGTA 3′
MYC F
5′ TCCACTCGGAAGGACTATCC 3′
MYC R
5′ TTACGCACAAGAGTTCCGTAG 3′

### Cell Staining and Fluorescence Activated Cell Sorting

For surface phenotyping and cell sorting the following fluorochrome (fluorescein isothiocyanate [FITC], AlexaFluor-488 [AF488], phycoerythrin [PE], or allophycocyanin [APC])—labeled monoclonal antibodies (mAbs) were used—all from Becton Dickinson Biosciences [BDB] San Jose, Calif.—unless otherwise indicated: antiCD9 FITC (M-L13), anti-SSEA-3 AF488 (ML-631) from eBiosciences (San Diego, Calif.); anti-CD221 PE (1H7), anti-CD49f PE (GoH3), anti-CD24 PE (ML5), anti-SSEA4 PE (MC813-70) from R&D (Minneapolis, Minn.); anti-CD90 APC(5E10), anti-CD29 APC (MAR4), anti-CD71 FITC (M-A712) and anti-ALP APC from R&D (Minneapolis, Minn.). The specificity of the staining was verified by the use of the matched isotype control mAbs. For the immunophenotype characterization a total of 10000 events were collected. Hoechst 33528 (H258) was included at 1 μg/mL in the final wash to detect dead cells. All analyses were performed on a Moflo cell sorter (DakoCytomation) applying Summit software.

qPCR Analysis of the Clones

Total mRNA was isolated using TRIZOL and 1 μg was used to synthesize cDNA using the Invitrogen Cloned AMV First-Strand cDNA synthesis kit. One μl of the reaction was used to quantify gene expression by qPCR using the following primers:

Oct4
Forward Primer
GGAGGAAGCTGACAACAATGAAA
Reverse Primer
GGCCTGCACGAGGGTTT
Sox2
Forward Primer
TGCGAGCGCTGCACAT
Reverse Primer
TCATGAGCGTCTTGGTTTTCC
Nanog
Forward Primer
ACAACTGGCCGAAGAATAGCA
Reverse Primer
GGTTCCCAGTCGGGTTCAC
CRIPTO
Forward Primer
CGGAACTGTGAGCACGATGT
Reverse Primer
GGGCAGCCAGGTGTCATG
Rex1
Forward Primer
CCTGCAGGCGGAAATAGAAC
Reverse Primer
GCACACATAGCCATCACATAAGG
Klf4
Forward Primer
CGAACCCACACAGGTGAGAA
Reverse Primer
GAGCGGGCGAATTTCCAT
c-myc
Forward Primer
AGGGTCAAGTTGGACAGTGTCA
Reverse Primer
TGGTGCATTTTCGGTTGTTG
Tbx5
Forward Primer
ATGTCAAGAATGCAAAGTAAAGAATATCC
Reverse Primer
GACTCGCTGCTGAAAGGACTGT
MEF2C
Forward Primer
CTGGCAACAGCAACACCTACA
Reverse Primer
GCTAGTGCAAGCTCCCAACTG
FoxA2
Forward Primer
CTGAAGCCGGAACACCACTAC
Reverse Primer
CGAGGACATGAGGTTGTTGATG
HNF4
Forward Primer
CTGCAGGCTCAAGAAATGCTT
Reverse Primer
TCATTCTGGACGGCTTCCTT
Sox17
Forward Primer
TGGCGCAGCAGAATCCA
Reverse Primer
CCACGACTTGCCCAGCAT
TUBB3
Forward Primer
GGCCAAGTTCTGGGAAGTCA
Reverse Primer
CGAGTCGCCCACGTAGTTG
Pax6
Forward Primer
GCTTCACCATGGCAAATAACC
Reverse Primer
GGCAGCATGCAGGAGTATGA
GAPDH
Forward Primer
GCACCGTCAAGGCTGAGAAC
Reverse Primer
AGGGATCTCGCTCCTGGAA
Trans-Oct4
Forward Primer
TGGACTACAAGGACGACGATGA
Reverse Primer
CAGGTGTCCCGCCATGA
Trans-Sox2
Forward Primer
GCTCGAGGTTAACGAATTCATGT
Reverse Primer
GCCCGGCGGCTTCA
Trans-Klf4
Forward Primer
TGGACTACAAGGACGACGATGA
Reverse Primer
CGTCGCTGACAGCCATGA
Trans-c-myc
Forward Primer
TGGACTACAAGGACGACGATGA
Reverse Primer
GTTCCTGTTGGTGAAGCTAACGT
Endo-Oct4
Forward Primer
GGGTTTTTGGGATTAAGTTCTTCA
Reverse Primer
GCCCCCACCCTTTGTGTT
Endo-Sox2
Forward Primer
CAAAAATGGCCATGCAGGTT
Reverse Primer
AGTTGGGATCGAACAAAAGCTATT
Endo-Klf4
Forward Primer
AGCCTAAATGATGGTGCTTGGT
Reverse Primer
TTGAAAACTTTGGCTTCCTTGTT
Endo-c-myc
Forward Primer
CGGGCGGGCACTTTG
Reverse Primer
GGAGAGTCGCGTCCTTGCT

For the analysis of expression of stem cell related genes, the Human Stem Cell RT2 profiler PCR array was used (SuperArray Biosciences Corporation) with 1 μg of total RNA following the manufacturers' directions. Expression values were normalized to the average expression of housekeeping genes. Values below 0.44 were considered to be not expressed and marked as 0, and then clustered using a Pearson correlation as a distance measure, and using pairwise complete linkage, as implemented in GenePattern in the HierarchicalClustering module. For graphical representation the Hierarchical Clustering Viewer from GenePattern was used, with coloring relative for each gene row.

Genomic DNA PCR Primers Used were:

qFLAGfw:
ATGGACTACAAGGACGACGATGAC
qOct4brv:
TCAGGCTGAGAGGTCTCCAA
qSox2rv:
TATAATCCGGGTGCTCCTTC
qKlf4rv:
CGTTGAACTCCTCGGTCTCT
qMycrv:
CAGCAGCTCGAATTTCTTCC

### Example 3

### Tripotent Capacity of KiPS

One of the key features of embryonic stem cells is their capacity to differentiate into all three germ cell layers. To test this in KiPS cells, embryoid bodies (KiPS4F1 and KiPS3F1) were generated followed by a variety of specific differentiation protocols. Differentiation into alpha-feto protein positive endoderm, alpha-actinin positive muscle cells (mesoderm) and beta3-tubulin positive neuronal cells (ectoderm) (FIGS. 3 a-d), representative of the main three germ layers, was clearly evident. Furthermore, highly efficient and full differentiation into dopaminergic neurons was achieved (FIGS. 3 e-f) showing the potential of KiPS cells for complete cellular differentiation, particularly important for transplantation purposes. In support of these in-vitro differentiation studies intratesticular teratomas in mice injected with KiPS4F1 cells were analysed. Histological evidence of all main three lineages was evident (FIGS. 3 g-j).

### Materials and Methods

### In Vitro Differentiation

Cells were trypsinized into a single cell suspension and resuspended in conditioned media. EB formation was induced by seeding 20,000-30,000 KiPS cells in 200 μl of conditioned media in each well of 96-well round bottom, low attachment plates and centrifuging the plates at 950 g for 5 min to aggregate the cells. After 3-4 days the EBs were transferred to 0.1% gelatine-coated glass chamber slides and cultured in differentiation medium (DMEM supplemented with 20% fetal bovine serum, 2 mM L-glutamine, 0.1 mM 2-mercaptoethanol, non-essential amino acids, and penicillin-streptomycin) for 2-3 weeks. The medium was changed every other day. For cardiomyocyte differentiation, KiPS cells were maintained on gelatin-coated plate in differentiated medium supplemented with 100 Mm ascorbic acid (Sigma).

Co-culture with the stromal cell line PA6 was used for KiPS differentiation into dopaminergic neurons. Briefly, after 10 days as a floating culture in N2B27 medium with FGF-2 (20 ng/ml), Sonic Hedgehog (0.1 ug/μl), and FGF8 (100 ng/ml), EBs were plated on PA6-feeder layer and maintained for 3-5 weeks in the absence of FGF2. The medium was changed every other day.

### Example 4

### KiPS Generation Efficiency

The generation of KiPS cells appeared to be much more efficient than that of fibroblast reprogramming. To investigate this in more detail, the timing of keratinocyte reprogramming was analysed during KiPS cell generation. Morphologically, nascent KiPS cell colonies could be identified as early as 10 days post-infection (6 days after seeding onto feeders, FIG. 1c and FIG. 7g). In contrast, colonies of iPS cells generated by retroviral transduction of human fibroblasts with the same 4 factors appear after 21-25 days post-infection (Takahashi, K. et al. (2007) Cell 131, 861-872; Yu, J. et al. (2007) Science 318, 1917-1920; Park, I. H. et al. (2008) Nature 451, 141-146 (2008)).

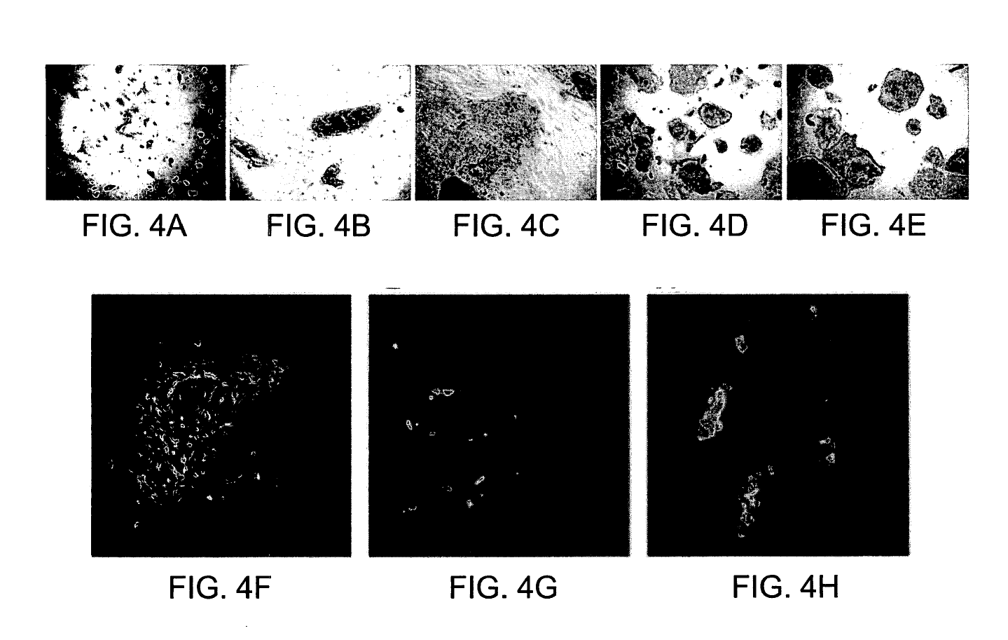

Importantly, at 10 days post-infection, most KiPS cell colonies already displayed strong AP activity, which appeared as a mosaic pattern of AP-positive and AP-negative cells. A similar pattern was seen in colonies at day 14, whereas after 17 or 21 days post-infection KiPS cell colonies were uniformly AP positive (FIG. 4a-e and FIG. 11). Other hES cell markers, such as SSEA4 and Tra-1-81 were also expressed in KiPS cell colonies in a similar manner as early as 14 days post-infection (FIGS. 4 f-h). Conversely, the amount of keratinocyte-specific proteins, such as keratin 14, gradually declined in KiPS cell colonies after 8, 10 and 12 days post-infection while being progressively displaced to the periphery of KiPS cell colonies, with restricted expression in single cells at day 14 (FIG. 11 and data not shown). These results indicate that the keratinocyte reprogramming of the invention appears to be faster than that of fibroblasts.

In addition to being faster, the keratinocyte reprogramming of the invention is also a more efficient process than that of fibroblasts. Thus, ˜400 KiPS cell colonies (379±52, n=3) were typically obtained from 50,000 infected keratinocytes (FIG. 4i), representing an overall reprogramming efficiency close to 1%. To test whether this high efficiency depended on the cell type, rather than the specific retroviruses or infection protocol utilized in this study, the equivalent retroviral supernatants were used to transduce primary dermal fibroblasts isolated from the same foreskin biopsy from which keratinocytes were obtained. At 31 days post-infection, 100-150 “granulated” colonies and around 4 hES cell-like AP-positive colonies (3.7±1.5, n=3; FIG. 4i) were obtained from 50,000 infected fibroblasts, or an overall efficiency of less than 0.01%, in agreement with previous reports. These results clearly show that the reprogramming model of human keratinocytes of the invention is, at least, 100-fold more efficient than that of fibroblasts known in the art. This difference in reprogramming ability may be, at least in part, explained by the rapid differentiation of non-reprogrammed keratinocytes when cultured in ES cell media, which would therefore act as a positive selection for reprogrammed keratinocytes.

However, (i) the fact that KiPS cells displayed fewer retroviral integrations (FIG. 9) than fibroblast-derived iPS cells, and (ii) that the expression levels of the transgenic factors in infected keratinocytes were lower than those of infected fibroblasts (data not shown), suggest that intrinsic differences between both cell types are important in accounting for the high reprogramming efficiency of keratinocytes.

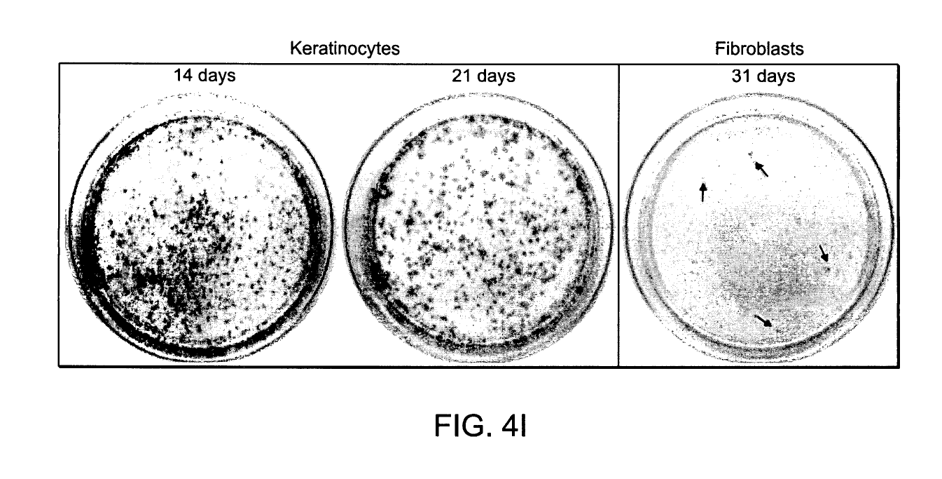

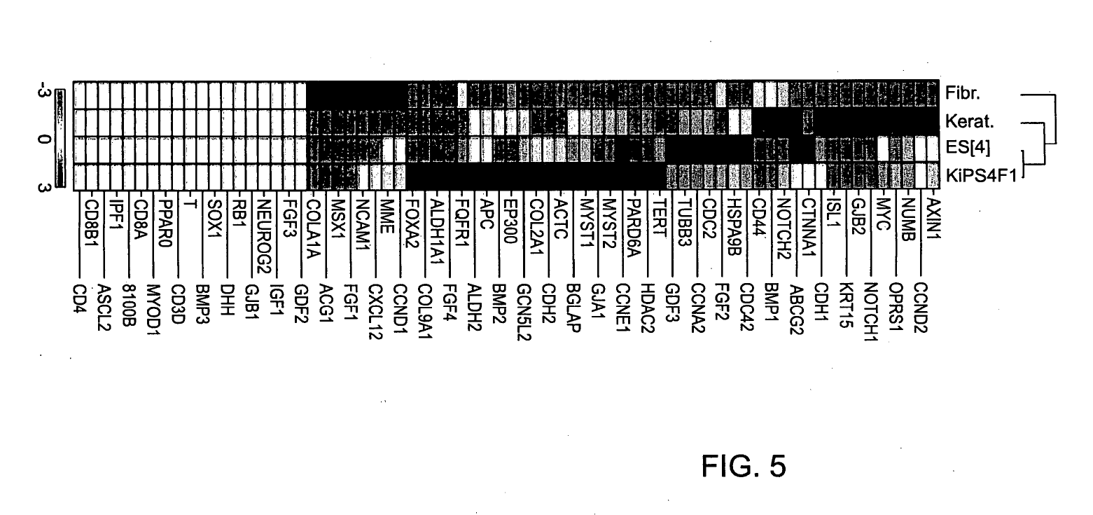

In the mouse, hepatocytes and gastric epithelial cells also appear to be more easily reprogrammed and require fewer retroviral integrations than fibroblasts, although the mechanism(s) responsible for this difference is unknown. Expression of Oct4, Nanog, Sox2, Cripto, or Rex1 was not detected in either keratinocytes or fibroblasts. However, keratinocytes displayed much higher (14 and 30 fold higher respectively) expression of c-Myc and Klf4 than fibroblasts (FIG. 10), showing that the elevated levels of endogenous expression of these factors render keratinocytes especially susceptible to reprogramming. In this respect, it has been shown that Klf4 blocks the proliferation-differentiation switch of basal keratinocytes, whereas c-Myc stimulates exit from the adult stem cell compartment to drive cells into a proliferative mode towards the epidermal and sebaceous gland lineage, while inducing major global histone modifications at the same time. Interestingly, c-Myc has also been shown to induce high levels of telomerase activity in keratinocytes as well as to repress terminal differentiation in response to a high-calcium switch, a circumstance that occurs in the reprogramming protocol when cells are changed from keratinocyte to ES cell culture media. It was also observed that keratinocytes, unlike fibroblasts, displayed high level of expression of stem cell markers, such as CD24 (FIG. 8), prompting the notion that the transcriptional profile of keratinocytes could be more similar to that of hES cells than that of fibroblasts. To explore this further, the expression levels of an array of genes related to stem cell identity, growth, or differentiation in keratinocytes, fibroblasts, hES cells, and KiPS cells were analysed by real-time RT-PCR. Gene expression values were clustered using a Pearson correlation as a distance measure, and compared among samples using pairwise complete linkage. Both analyses clearly indicate that the transcriptional profile of keratinocytes is significantly more similar to that of hES cells and KiPS cells than that of fibroblasts (FIG. 5).

### Example 5

KiPS Generation from Minute Amounts of Biological Samples

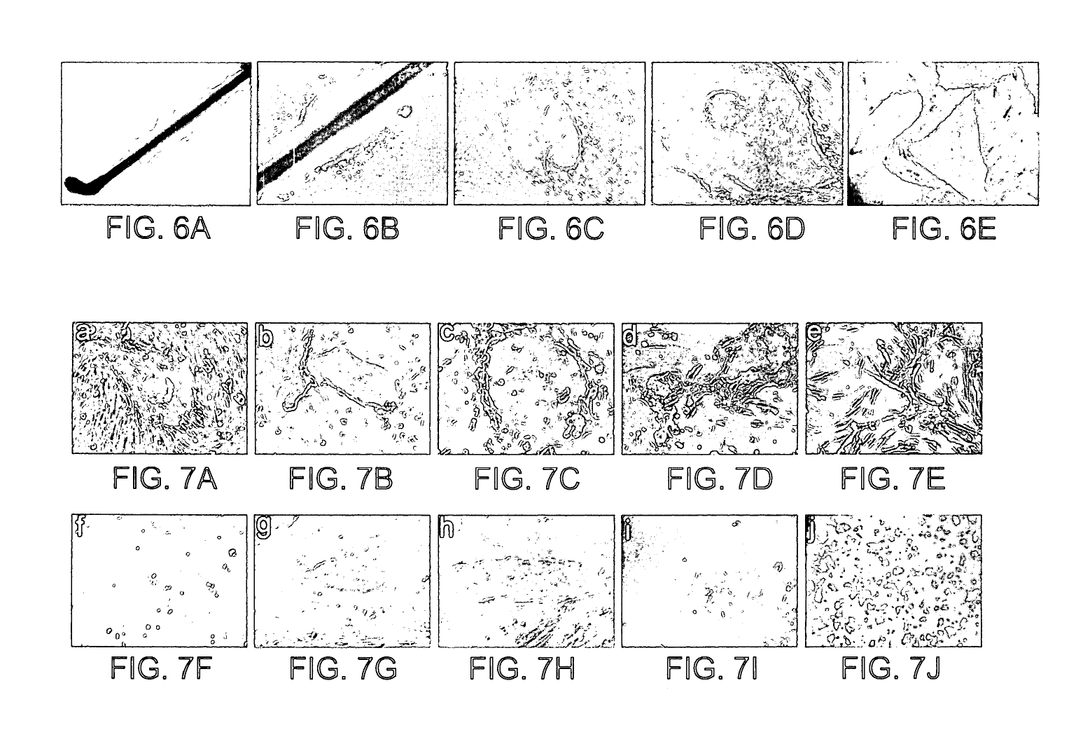

The ability of KiPS cells to be established from minute amounts of biological samples was determined. In one example, a single hair plucked from a 30-year-old woman was used. Upon plating, keratinocytes proliferated out of the outer root sheet area, whereas no cells were observed growing from the bulb area (FIG. 6a-b and FIG. 4i). Keratinocytes isolated in this way were split once and then subjected to the reprogramming protocol of the invention, which resulted in the successful generation of KiPS cell colonies (FIGS. 6 c-d). Such colonies could be expanded after mechanical picking, stained strongly for AP activity, the expression of all pluripotency markers and capacity to differentiate, and showed identical colony morphology and growth characteristics to hES cells and KiPS cells from foreskin keratinocytes (FIG. 6 e and data not shown).

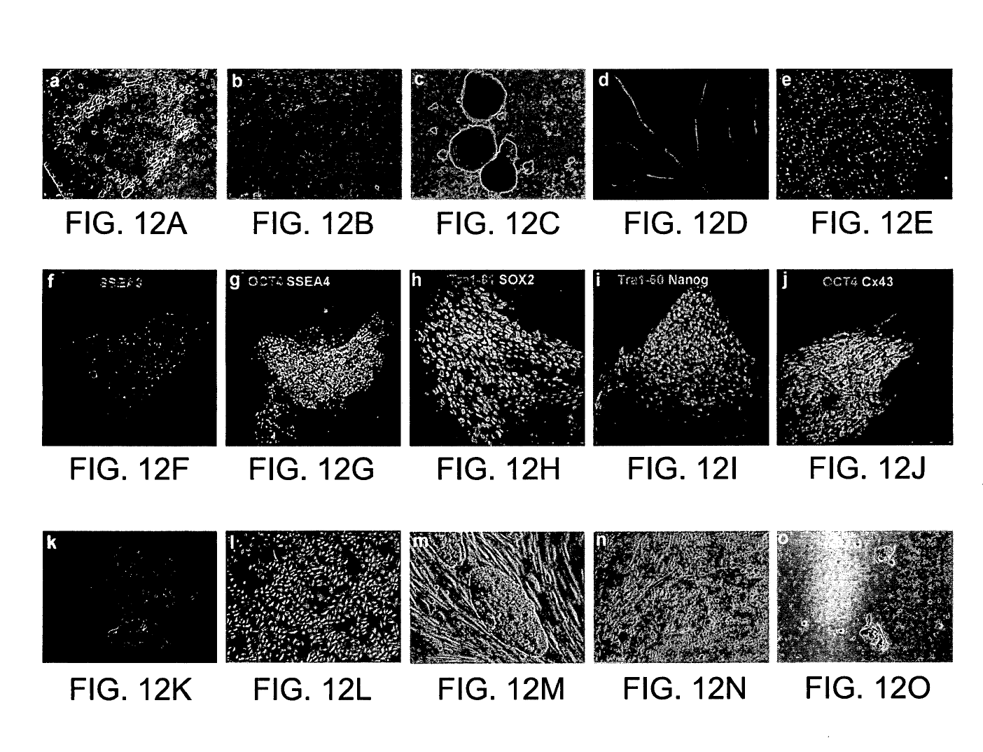

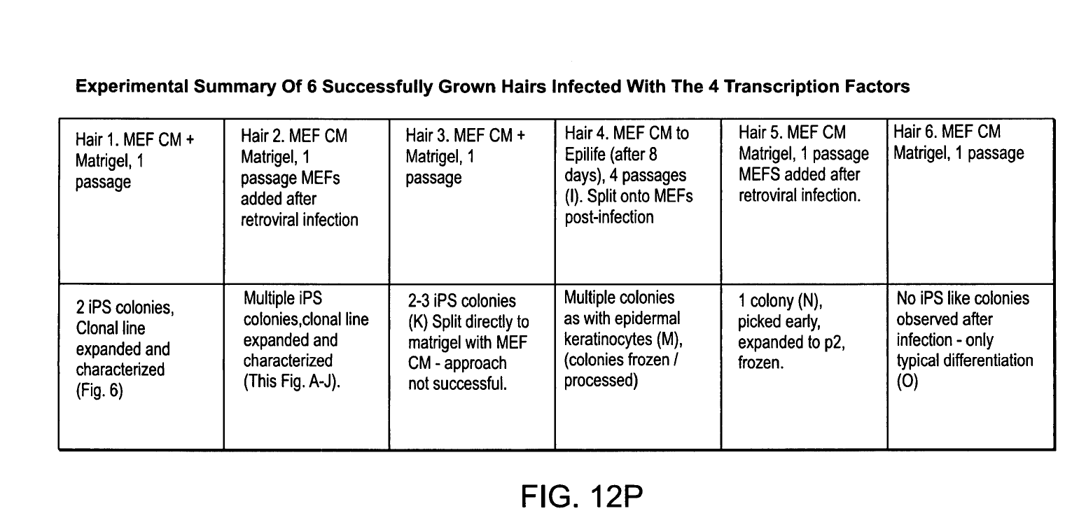

Additional methods have been used successfully to generate KiPS from individuals. In addition to the results presented in FIG. 6, hair derived from five additional subjects (four female, one male) was used successfully as a source of KiPS (see FIG. 12). A variety of KiPS cell derivation-strategies were used. In one experiment, irradiated MEFs were added to a dish directly after retroviral infection (hair sample 2). This improved growth of the reprogrammed colonies (FIG. 12a). These colonies were picked and a line was established successfully (FIG. 12b). This line was used to derive embyoid bodies (FIG. 12c) and also differentiates spontaneously into a variety of cell types some of which have been identified, including endothelial like cells positive for von Willebrand Factor (FIG. 12d), as well as colonies positive for FoxA2 and Tuj1 (FIG. 12e). The line was tested for expression of all the typical pluirpotency markers including SSEA3, SSEA4, Oct4, Tra 1-81, Tra 1-60, Sox2, Nanog and Cx43, all of which were strongly positive as in the initial hair KiPS (FIG. 12f-j). The cells of hair sample 3, as with sample 2, provided for identifiable iPS like colonies that grew out of a matrigel coated dish without feeders (FIG. 12k). These cells were trypsinized directly onto another matrigel-covered dish. A few days prior to splitting the cells growing out of the hair, cells derived from hair sample 4 were grown in the presence of Epilife. Addition of Epilife caused an increase in the proliferation of these cells. These cells also adopted a morphology identical to epidermally derived keratinocytes grown under the same conditions (FIG. 12l).

These cells were successfully passaged four times and successful generation of typical KiPS colonies was achieved following the procedure used on foreskin samples (FIG. 12m). Sample 5 was treated as sample 2, although only one typical KiPS colony was positively identified (FIG. 12n). This colony was picked and frozen at an early stage. Sample 6 (male) did not display any typical KiPS colonies but rather differentiated into clumps of keratinocytes (FIG. 12o) as in uninfected hair and epidermal samples. This may be due to onset of keratinocyte differentiation before retroviral infection, as this is highly dependent on seeding density of keratinocytes which varied widely (1 hair per 35 mm dish).

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. An isolated keratinocyte or keratinocyte population transfected with the genes selected from the group which comprises: an Oct4 family gene; Sox2 family gene and a Klf family gene.

2. The keratinocyte or keratinocyte population according to claim 1 further transfected with a c-Myc family gene.

3. The keratinocyte or keratinocyte population according to any of claim 1, wherein the genes also comprise a promoter to modulate its expression.

4. The keratinocyte or keratinocyte population according to claim 1, wherein said keratinocyte or keratinocyte population is of human origin.

5. The keratinocyte or keratinocyte population according to claim 1, wherein at least one of the genes is cloned into a murine stem cell virus (MSCV) derived retroviral vector.

6. An isolated keratinocyte induced stem (KiPS) cell or KiPS cell population derived from any of the keratinocyte or keratinocyte population of claim 1.

7. A KiPS cell or KiPS cell population of claim 6 characterized by the expression of one or more of the following markers Nanog, Oct 4, Sox2, Rex1, Cripto, Connexin43, IGF-1 receptor, SSEA4, SSEA3, Tra-1-61 and Tra-1-81.

8. The KiPS cell or KiPS cell population of claim 6 further characterized by the expression of one or more of the following markers: AP marker, CD24, CD90, CD29, CD9 and CD49f.

9. A Method for obtaining a KiPS cell or a KiPS cell population according to claim 1 which comprises transfecting a polynucleotide or polynucleotides into an isolated keratinocyte or isolated keratinocyte population, wherein said polynucleotide or polynucleotides encoding the following group of genes: an Oct4 family gene; a Sox2 family gene, and a Klf family gene.

10. The method of claim 9 wherein the polynucleotide or polynucleotides further encodes a c-Myc family gene.

11. The method for obtaining a KiPS cell or a KiPS cell population according to claim 9, wherein said polynucleotide or polynucleotides are cloned into a vector.

12. The method of claim 11 wherein the vector is the murine stem cell virus (MSCV) derived retroviral vector.

13. The method according to claim 9 wherein the isolated keratinocyte or isolated keratinocyte population is obtained by plating a hair or a root hair in a culture media which promotes keratinocyte proliferation.

14. A method for obtaining iPS cells which comprises the following steps:
a. Positively selecting an isolated somatic cell or an isolated somatic cell population by the comparison of the expression level of any of the markers c-Myc or Klf4 with an isolated fibroblast cell or an isolated fibroblast population, wherein said positive selection is made, where the expression level of either of said markers is at least 10 fold higher in comparison with the expression level in the isolated fibroblast cell or the isolated fibroblast population,
b. Transfecting the selected isolated somatic cell or the selected isolated somatic population selected in step a) with a polynucleotide or polynucleotides encoding the following group of genes: an Oct4 family gene; Sox2 family gene, a Klf family gene, and
c. Placing the transfected isolated somatic cell or isolated cell population in an appropriate dedifferentiating medium.

15. The method of claim 14 wherein the group of step b) also comprises a c-Myc family gene.

16. The method of claim 14, wherein the isolated somatic cell or the isolated somatic cell population is human.

17. A composition comprising the keratinocyte population of claim 1 for use as a medicament.

18. (canceled)

19. A composition comprising the KiPS cell population of claim 7 for use as a medicament.

20. (canceled)

21. (canceled)

22. (canceled)

23. (canceled)

24. (canceled)

25. A method of treating a patient with a defect in a tissue with mesenchymal origin, ectodermal origin or endodermal origin comprising administering to a patient in need thereof a therapeutically effective amount of the keratinocyte or keratinocyte population of claim 1.

26. A method of treating a patient with a defect in a tissue with mesenchymal origin, ectodermal origin or endodermal origin comprising administering to a patient in need thereof a therapeutically effective amount of KiPS cells or the KiPS cells population of claim 7.

27. (canceled)


SEQUENCE LISTING (59 sequences)

SEQ ID NO: 1 - DNA, 20 bp, Artificial Sequence
  Description of Artificial Sequence Synthetic primer
agtacaactccatgaccagc

SEQ ID NO: 2 - DNA, 19 bp, Artificial Sequence
  Description of Artificial Sequence Synthetic primer
tcacatgtgtgagaggggc

SEQ ID NO: 3 - DNA, 19 bp, Artificial Sequence
  Description of Artificial Sequence Synthetic primer
taagcttccaaggccctcc

SEQ ID NO: 4 - DNA, 19 bp, Artificial Sequence
  Description of Artificial Sequence Synthetic primer
ctcctccgggttttgctcc

SEQ ID NO: 5 - DNA, 20 bp, Artificial Sequence
  Description of Artificial Sequence Synthetic primer
aattacccatccttcctgcc

SEQ ID NO: 6 - DNA, 23 bp, Artificial Sequence
  Description of Artificial Sequence Synthetic primer
ttaaaaatgcctcttcatgtgta

SEQ ID NO: 7 - DNA, 20 bp, Artificial Sequence
  Description of Artificial Sequence Synthetic primer
tccactcggaaggactatcc

SEQ ID NO: 8 - DNA, 21 bp, Artificial Sequence
  Description of Artificial Sequence Synthetic primer
ttacgcacaagagttccgtag

SEQ ID NO: 9 - DNA, 23 bp, Artificial Sequence
  Description of Artificial Sequence Synthetic primer
ggaggaagctgacaacaatgaaa

SEQ ID NO: 10 - DNA, 17 bp, Artificial Sequence
  Description of Artificial Sequence Synthetic primer
ggcctgcacgagggttt

SEQ ID NO: 11 - DNA, 16 bp, Artificial Sequence
  Description of Artificial Sequence Synthetic primer
tgcgagcgctgcacat

SEQ ID NO: 12 - DNA, 21 bp, Artificial Sequence
  Description of Artificial Sequence Synthetic primer
tcatgagcgtcttggttttcc

SEQ ID NO: 13 - DNA, 21 bp, Artificial Sequence
  Description of Artificial Sequence Synthetic primer
acaactggccgaagaatagca

SEQ ID NO: 14 - DNA, 19 bp, Artificial Sequence
  Description of Artificial Sequence Synthetic primer
ggttcccagtcgggttcac

SEQ ID NO: 15 - DNA, 20 bp, Artificial Sequence
  Description of Artificial Sequence Synthetic primer
cggaactgtgagcacgatgt

SEQ ID NO: 16 - DNA, 18 bp, Artificial Sequence
  Description of Artificial Sequence Synthetic primer
gggcagccaggtgtcatg

SEQ ID NO: 17 - DNA, 20 bp, Artificial Sequence
  Description of Artificial Sequence Synthetic primer
cctgcaggcggaaatagaac

SEQ ID NO: 18 - DNA, 23 bp, Artificial Sequence
  Description of Artificial Sequence Synthetic primer
gcacacatagccatcacataagg

SEQ ID NO: 19 - DNA, 20 bp, Artificial Sequence
  Description of Artificial Sequence Synthetic primer
cgaacccacacaggtgagaa

SEQ ID NO: 20 - DNA, 18 bp, Artificial Sequence
  Description of Artificial Sequence Synthetic primer
gagcgggcgaatttccat

SEQ ID NO: 21 - DNA, 22 bp, Artificial Sequence
  Description of Artificial Sequence Synthetic primer
agggtcaagttggacagtgtca

SEQ ID NO: 22 - DNA, 20 bp, Artificial Sequence
  Description of Artificial Sequence Synthetic primer
tggtgcattttcggttgttg

SEQ ID NO: 23 - DNA, 29 bp, Artificial Sequence
  Description of Artificial Sequence Synthetic primer
atgtcaagaatgcaaagtaaagaatatcc

SEQ ID NO: 24 - DNA, 22 bp, Artificial Sequence
  Description of Artificial Sequence Synthetic primer
gactcgctgctgaaaggactgt

SEQ ID NO: 25 - DNA, 21 bp, Artificial Sequence
  Description of Artificial Sequence Synthetic primer
ctggcaacagcaacacctaca

SEQ ID NO: 26 - DNA, 21 bp, Artificial Sequence
  Description of Artificial Sequence Synthetic primer
gctagtgcaagctcccaactg

SEQ ID NO: 27 - DNA, 21 bp, Artificial Sequence
  Description of Artificial Sequence Synthetic primer
ctgaagccggaacaccactac

SEQ ID NO: 28 - DNA, 22 bp, Artificial Sequence
  Description of Artificial Sequence Synthetic primer
cgaggacatgaggttgttgatg

SEQ ID NO: 29 - DNA, 21 bp, Artificial Sequence
  Description of Artificial Sequence Synthetic primer
ctgcaggctcaagaaatgctt

SEQ ID NO: 30 - DNA, 20 bp, Artificial Sequence
  Description of Artificial Sequence Synthetic primer
tcattctggacggcttcctt

SEQ ID NO: 31 - DNA, 17 bp, Artificial Sequence
  Description of Artificial Sequence Synthetic primer
tggcgcagcagaatcca

SEQ ID NO: 32 - DNA, 18 bp, Artificial Sequence
  Description of Artificial Sequence Synthetic primer
ccacgacttgcccagcat

SEQ ID NO: 33 - DNA, 20 bp, Artificial Sequence
  Description of Artificial Sequence Synthetic primer
ggccaagttctgggaagtca

SEQ ID NO: 34 - DNA, 19 bp, Artificial Sequence
  Description of Artificial Sequence Synthetic primer
cgagtcgcccacgtagttg

SEQ ID NO: 35 - DNA, 21 bp, Artificial Sequence
  Description of Artificial Sequence Synthetic primer
gcttcaccatggcaaataacc

SEQ ID NO: 36 - DNA, 20 bp, Artificial Sequence
  Description of Artificial Sequence Synthetic primer
ggcagcatgcaggagtatga

SEQ ID NO: 37 - DNA, 20 bp, Artificial Sequence
  Description of Artificial Sequence Synthetic primer
gcaccgtcaaggctgagaac

SEQ ID NO: 38 - DNA, 19 bp, Artificial Sequence
  Description of Artificial Sequence Synthetic primer
agggatctcgctcctggaa

SEQ ID NO: 39 - DNA, 22 bp, Artificial Sequence
  Description of Artificial Sequence Synthetic primer
tggactacaaggacgacgatga

SEQ ID NO: 40 - DNA, 17 bp, Artificial Sequence
  Description of Artificial Sequence Synthetic primer
caggtgtcccgccatga

SEQ ID NO: 41 - DNA, 23 bp, Artificial Sequence
  Description of Artificial Sequence Synthetic primer
gctcgaggttaacgaattcatgt

SEQ ID NO: 42 - DNA, 14 bp, Artificial Sequence
  Description of Artificial Sequence Synthetic primer
gcccggcggcttca

SEQ ID NO: 43 - DNA, 22 bp, Artificial Sequence
  Description of Artificial Sequence Synthetic primer
tggactacaaggacgacgatga

SEQ ID NO: 44 - DNA, 18 bp, Artificial Sequence
  Description of Artificial Sequence Synthetic primer
cgtcgctgacagccatga

SEQ ID NO: 45 - DNA, 22 bp, Artificial Sequence
  Description of Artificial Sequence Synthetic primer
tggactacaaggacgacgatga

SEQ ID NO: 46 - DNA, 23 bp, Artificial Sequence
  Description of Artificial Sequence Synthetic primer
gttcctgttggtgaagctaacgt

SEQ ID NO: 47 - DNA, 24 bp, Artificial Sequence
  Description of Artificial Sequence Synthetic primer
gggtttttgggattaagttcttca

SEQ ID NO: 48 - DNA, 18 bp, Artificial Sequence
  Description of Artificial Sequence Synthetic primer
gcccccaccctttgtgtt

SEQ ID NO: 49 - DNA, 20 bp, Artificial Sequence
  Description of Artificial Sequence Synthetic primer
caaaaatggccatgcaggtt

SEQ ID NO: 50 - DNA, 24 bp, Artificial Sequence
  Description of Artificial Sequence Synthetic primer
agttgggatcgaacaaaagctatt

SEQ ID NO: 51 - DNA, 22 bp, Artificial Sequence
  Description of Artificial Sequence Synthetic primer
agcctaaatgatggtgcttggt

SEQ ID NO: 52 - DNA, 23 bp, Artificial Sequence
  Description of Artificial Sequence Synthetic primer
ttgaaaactttggcttccttgtt

SEQ ID NO: 53 - DNA, 15 bp, Artificial Sequence
  Description of Artificial Sequence Synthetic primer
cgggcgggcactttg

SEQ ID NO: 54 - DNA, 19 bp, Artificial Sequence
  Description of Artificial Sequence Synthetic primer
ggagagtcgcgtccttgct

SEQ ID NO: 55 - DNA, 24 bp, Artificial Sequence
  Description of Artificial Sequence Synthetic primer
atggactacaaggacgacgatgac

SEQ ID NO: 56 - DNA, 20 bp, Artificial Sequence
  Description of Artificial Sequence Synthetic primer
tcaggctgagaggtctccaa

SEQ ID NO: 57 - DNA, 20 bp, Artificial Sequence
  Description of Artificial Sequence Synthetic primer
tataatccgggtgctccttc

SEQ ID NO: 58 - DNA, 20 bp, Artificial Sequence
  Description of Artificial Sequence Synthetic primer
cgttgaactcctcggtctct

SEQ ID NO: 59 - DNA, 20 bp, Artificial Sequence
  Description of Artificial Sequence Synthetic primer
cagcagctcgaatttcttcc